(19) United States
(12) Patent Application Publication
(10) Pub. No.: US 2021/0107921 A1
(43) Pub. Date: Apr. 15, 2021

(54) HETEROCYCLIC COMPOUNDS

(71) Applicant: Hoffmann-La Roche Inc., Little Falls, NJ (US)

(72) Inventors: Joerg BENZ, Basel (CH); Luca GOBBI, Basel (CH); Uwe GRETHER, Basel (CH); Steven Paul HANLON, Basel (CH); Benoit HORNSPERGER, Basel (CH); Carsten KROLL, Basel (CH); Bernd KUHN, Basel (CH); Martin KURATLI, Basel (CH); Guofu LIU, Hubei (CN); Fionn O`HARA, Basel (CH); Hans RICHTER, Basel (CH); Martin RITTER, Basel (CH)

(73) Assignee: Hoffmann-La Roche Inc., Little Falls, NJ (US)

(21) Appl. No.: 17/026,619

(22) Filed: Sep. 21, 2020

(30) Foreign Application Priority Data
Sep. 23, 2019 (EP) 19198974.8
Aug. 14, 2020 (CN) PCT/CN2020/109184

(51) Int. Cl. C07D 498/04

(52) U.S. Cl. CPC C07D 498/04

(57) ABSTRACT

The invention provides new heterocyclic compounds having the general formula (I)

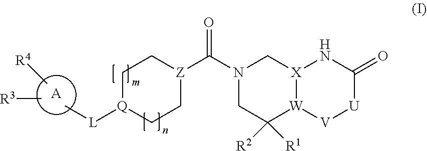

wherein A, L, Q, U, V, W, X, Z, m, n, and R¹ to R⁴ are as described herein, compositions including the compounds, processes of manufacturing the compounds and methods of using the compounds.

### CROSS-REFERENCE TO RELATED APPLICATIONS

This application claims priority to International Application No. PCT/CN2020/109184, filed Aug. 14, 2020, and EP Application No. 19198974.8, filed Sep. 23, 2019, the disclosure of each of which is incorporated herein by reference in its entirety.

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28.). In the brain, the main endocannabinoid, 2-arachidonoylglycerol (2-AG), is produced by diacylglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809.). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798.). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A2 (PLA2) enzymes have been viewed as the principal source of AA, but cPLA₂-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-a) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403.). Importantly, genetic and/or pharmacological disruption of MAGL activity also blocks LPS-induced activation of microglial cells in the brain (Nomura, D. K., et al., Science 2011, 334, 809.).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37.). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809.), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424.) and on mental disorders, such as depression in chronic stress models (Thong P. et al., Neuropsychopharmacology 2014, 39, 1763.).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163.). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421.). Inhibition of MAGL was also shown to promote remyelination and functional recovery in a mouse model of progressive multiple sclerosis (Feliu A. et al., Journal of Neuroscience 2017, 37 (35), 8385.).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140(1), 49-61; Nomura D K et al., Chem. Biol. 2011, 18(7), 846-856, Jinlong Yin et al, Nature Communications 2020, 11, 2978).

The endocannabinoid system is also involved in many gastrointestinal physiological and physiopathological actions (Marquez L. et al., PLoS One 2009, 4(9), e6893). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright K. et al., Gastroenterology 2005, 129(2), 437-453; Duncan, M. et al., Aliment Pharmacol Ther 2005, 22(8), 667-683). Activation of CB1 produces anti-emetic, anti-motility, and anti-inflammatory effect, and help to modulate pain (Perisetti, A. et al., Ann Gastroenterol 2020, 33(2), 134-144). CB2 receptors are expressed in immune cells such as plasma cells and macrophages, in the lamina propria of the GI tract (Wright K. et al., Gastroenterology 2005, 129(2), 437-453), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez L. et al., PLoS One 2009, 4(9), e6893) and 2-AG levels are increased in plasma of IBD patients (Grill, M. et al., Sci Rep 2019, 9(1), 2358). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez L. et al., PLoS One 2009, 4(9), e6893). Furthermore, MAGL inhibition improves gut wall integrity and intestinal permeability via a CB1 driven MoA (Wang, J. et al., Biochem Biophys Res Commun 2020, 525(4), 962-967).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides a compound of formula (I), or a pharmaceutically acceptable salt thereof,

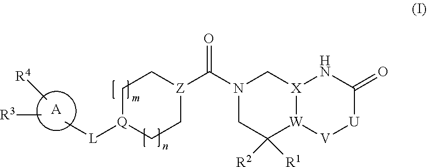

wherein A, L, Q, U, V, W, X, Z, m, n, and R¹ to R⁴ are as described herein.

In one aspect, the present invention provides a process of manufacturing the compounds of formula (I) described herein, comprising:
(a) reacting an amine of formula 2, wherein m, n, Q, L, A, R³ and R⁴ are as described herein,

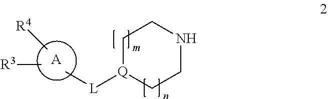

with a carboxylic acid 3a, wherein U, V, W, X, R¹ and R² are as described herein

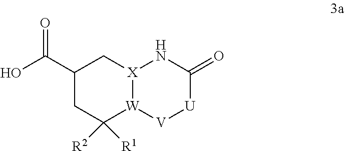

in the presence of a coupling reagent, and optionally in the presence of a base; or
(b) reacting an amine of formula 2, wherein m, n, Q, L, A, R³ and R⁴ are as described herein,

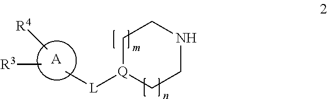

with a carboxylic acid chloride 3b, wherein U, V, W, X, R¹ and R² are as described herein

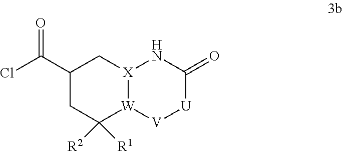

in the presence of a base; or
(c) reacting a first amine of formula 1, wherein U, V, W, X, R¹ and R² are as described herein,

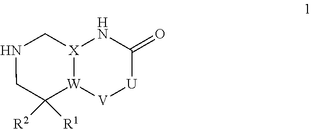

with a second amine 2, wherein A, L, m, n, Q, R³ and R⁴ are as described herein

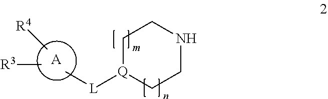

in the presence of a base and a urea forming reagent,
   to form said compound of formula (I).

In a further aspect, the present invention provides a compound of formula (I) as described herein, when manufactured according to the processes described herein.

In a further aspect, the present invention provides a compound of formula (I) as described herein, for use as therapeutically active substance.

In a further aspect, the present invention provides a pharmaceutical composition comprising a compound of formula (I) as described herein and a therapeutically inert carrier.

In a further aspect, the present invention provides the use of a compound of formula (I) as described herein or of a pharmaceutical composition described herein for inhibiting monoacylglycerol lipase (MAGL) in a mammal.

In a further aspect, the present invention provides the use of a compound of formula (I) as described herein or of a pharmaceutical composition described herein for the treatment or prophylaxis of neuroinflammation, neurodegenerative diseases, pain, cancer, mental disorders and/or inflammatory bowel disease in a mammal.

In a further aspect, the present invention provides the use of a compound of formula (I) as described herein or of a pharmaceutical composition described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁₋₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl and cycloheptyl. A particularly preferred example of cycloalkyl is cyclopropyl.

The terms “heterocyclyl” and “heterocycloalkyl” are used herein interchangeably and refer to a saturated or partly unsaturated mono- or bicyclic, preferably monocyclic ring system of 3 to 10 ring atoms, preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of monocyclic heterocyclyl groups include azetidin-3-yl, azetidin-2-yl, oxetan-3-yl, oxetan-2-yl, 1-piperidyl, 2-piperidyl, 3-piperidyl, 4-piperidyl, 2-oxopyrrolidin-1-yl, 2-oxopyrrolidin-3-yl, 5-oxopyrrolidin-2-yl, 5-oxopyrrolidin-3-yl, 2-oxo-1-piperidyl, 2-oxo-3-piperidyl, 2-oxo-4-piperidyl, 6-oxo-2-piperidyl, 6-oxo-3-piperidyl, morpholino, morpholin-2-yl and morpholin-3-yl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic.

Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic or bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from 0, S and N. Most preferably, “heteroaryl” refers to a 5-10 membered heteroaryl comprising 1 to 2 heteroatoms independently selected from O, S and N. Some preferred, yet non-limiting examples of heteroaryl include thiazolyl (e.g. thiazol-2-yl); oxazolyl (e.g. oxazol-2-yl); 5,6-dihydro-4H-cyclopenta[d]thiazol-2-yl; 1,2,4-oxadiazol-5-yl; pyridyl (e.g. 2-pyridyl); pyrazolyl (e.g. pyrazol-1-yl); imidazolyl (e.g. imidazole-1-yl); benzoxazolyl (e.g. benzoxazol-2-yl) and oxazolo[5,4-c]pyridin-2-yl.

The term “hydroxy” refers to an —OH group.

The term “cyano” refers to a —CN (nitrile) group.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl (CF₃) and trifluoroethyl (e.g. 2,2,2-trifluoroethyl).

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “aryloxy” refers to an aryl group, as previously defined, attached to the parent molecular moiety via an oxygen atom. A preferred, yet non-limiting example of aryloxy is phenoxy.

The term “cycloalkyloxy” refers to a cycloalkyl group, as previously defined, attached to the parent molecular moiety via an oxygen atom. A preferred, yet non-limiting example of cycloalkyloxy is cyclopropoxy.

The term “heteroaryloxy” refers to a heteroaryl group, as previously defined, attached to the parent molecular moiety via an oxygen atom. A preferred, yet non-limiting example of heteroaryloxy is pyridyloxy (e.g., 2-pyridyloxy, 3-pyridyloxy or 4-pyridyloxy).

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride salts.

The term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates. Examples of pharmaceutically acceptable prodrug types are described in Higuchi and Stella, Pro-drugs as Novel Delivery Systems, Vol. 14 of the A.C.S. Symposium Series, and in Roche, ed., Bioreversible Carriers in Drug Design, American Pharmaceutical Association and Pergamon Press, 1987.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

The term “urea forming reagent” refers to a chemical compound that is able to render a first amine to a species that will react with a second amine, thereby forming an urea derivative. Non-limiting examples of urea forming reagents include bis(trichloromethyl) carbonate, phosgene, trichloromethyl chloroformate, (4-nitrophenyl)carbonate and 1,1′-carbonyldiimidazole. The urea forming reagents described in G. Sartori et al., Green Chemistry 2000, 2, 140 are incorporated herein by reference.

The compounds of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates. In a preferred embodiment, the compound of formula (I) according to the invention is a cis-enantiomer of formula (Ia) or (Ib), respectively, as described herein.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and psychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect (A1), the present invention provides a compound of formula (I)

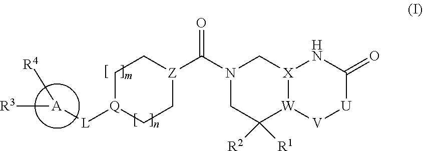

or a pharmaceutically acceptable salt thereof,
wherein:
(i) U is CH₂;
   V is O;
   W and X are both CH;
   R¹ is selected from halogen and C₁₋₆-alkyl; and
   R² is selected from hydrogen, halogen, and C₁₋₆-alkyl; or
   R¹ and R², taken together with the carbon atom to which they are attached, form a C₃-C₁₀-cycloalkyl; or
(ii) U is CH₂;
   V is O;
   W is CR^{w};
   X is CH;
   R^{w} is selected from halogen, and C₁₋₆-alkyl;
   R¹ and R² are independently selected from hydrogen, halogen, and C₁₋₆-alkyl; or
   R¹ and R², taken together with the carbon atom to which they are attached, form a C₃-C₁₀-cycloalkyl; or
(iii) U is CH₂;
   V is O;
   W and X together form a group C═C; and
   R¹ and R² are independently selected from hydrogen, halogen, and C₁₋₆-alkyl; or
   R¹ and R², taken together with the carbon atom to which they are attached, form a C₃-C₁₀-cycloalkyl; or
(iv) U is CH₂;
   V is selected from NH, CH₂, S, S═O, SO₂, CHOH, CHF, and CF₂;
   (a) W is CR^{w}; and
      X is CH; or
   (b) W and X together form a group C═C;
   R^{w} is selected from hydrogen, halogen, and C₁₋₆-alkyl;
   R¹ and R² are independently selected from hydrogen, halogen, and C₁₋₆-alkyl; or
   R¹ and R², taken together with the carbon atom to which they are attached, form a C₃-C₁₀-cycloalkyl; or
(v) U and V together form a group C═C;
   W and X together form a group C═C; and
   R¹ and R² are independently selected from hydrogen, halogen, and C₁₋₆-alkyl; or
   R¹ and R², taken together with the carbon atom to which they are attached, form a C₃-C₁₀-cycloalkyl; or
(vi) U is CH₂;
   V is O;
   W is CH;
   X is C—OH; and
   R¹ and R² are independently selected from hydrogen, halogen, and C₁₋₆-alkyl; or
   R¹ and R², taken together with the carbon atom to which they are attached, form a C₃-C₁₀-cycloalkyl;
m and n are both 0; or
m and n are both 1;
Z is CH or N;
Q is CR^{q} or N;
R^{q} is selected from hydrogen, halogen, hydroxy, halo-C₁₋₆-alkyl, and C₁₋₆-alkyl.
L is selected from a covalent bond, —CHR⁵—, —O—, —OCH₂—, —CH₂O—, —CH₂OCH₂—, —CF₂CH₂—, and —CH₂CF₂—;
A is selected from C₆-C₁₄-aryl, 5- to 14-membered heteroaryl, and 3- to 14-membered heterocyclyl;
R³ and R⁴ are independently selected from hydrogen, halogen, SF₅, cyano, C₁₋₆-alkyl, C₁₋₆-alkoxy, halo-C₁₋₆-alkyl, halo-C₁₋₆-alkoxy, C₆-C₁₄-aryl, C₃-C₁₀-cycloalkyl, 5-14-membered heteroaryl, C₆-C₁₄-aryloxy, C₃-C₁₀-cycloalkyloxy, and 5-14-membered heteroaryloxy, wherein said C₆-C₁₄-aryl, C₃-C₁₀-cycloalkyl, 5-14-membered heteroaryl, C₆-C₁₄-aryloxy, C₃-C₁₀-cycloalkyloxy, and 5-14-membered heteroaryloxy, are optionally substituted with 1-2 substituents selected from halogen, C₁₋₆-alkyl, and halo-C₁₋₆-alkyl; and

R⁵ is selected from hydrogen and C₆-C₁₄-aryl.

In a second aspect (A2), the present invention provides a compound of formula (I), or a pharmaceutically acceptable salt thereof, wherein:
(i) U is CH₂;
   V is O;
   W and X are both CH;
   R¹ is selected from halogen and C₁₋₆-alkyl; and
   R² is selected from hydrogen, halogen, and C₁₋₆-alkyl; or
   R¹ and R², taken together with the carbon atom to which they are attached, form a C₃-C₁₀-cycloalkyl; or
(ii) U is CH₂;
   V is O;
   W is CR^{w};
   X is CH;
   R^{w} is selected from halogen, and C₁₋₆-alkyl;
   R¹ and R² are independently selected from hydrogen, halogen, and C₁₋₆-alkyl; or
   R¹ and R², taken together with the carbon atom to which they are attached, form a C₃-C₁₀-cycloalkyl; or
(iii) U is CH₂;
   V is O;
   W and X together form a group C═C; and
   R¹ and R² are independently selected from hydrogen, halogen, and C₁₋₆-alkyl; or
   R¹ and R², taken together with the carbon atom to which they are attached, form a C₃-C₁₀-cycloalkyl; or
(iv) U is CH₂;
   V is selected from NH, CH₂, S, S═O, SO₂, CHOH, CHF, and CF₂;
   (c) W is CR^{w}; and
      X is CH; or
   (d) W and X together form a group C═C;
   R^{w} is selected from hydrogen, halogen, and C₁₋₆-alkyl;
   R¹ and R² are independently selected from hydrogen, halogen, and C₁₋₆-alkyl; or
   R¹ and R², taken together with the carbon atom to which they are attached, form a C₃-C₁₀-cycloalkyl; or
(v) U and V together form a group C═C;
   W and X together form a group C═C; and
   R¹ and R² are independently selected from hydrogen, halogen, and C₁₋₆-alkyl; or
   R¹ and R², taken together with the carbon atom to which they are attached, form a C₃-C₁₀-cycloalkyl;
m and n are both 0; or
m and n are both 1;
Z is CH or N;
Q is CR^{q} or N;
R^{q} is selected from hydrogen, halogen, hydroxy, halo-C₁₋₆-alkyl, and C₁₋₆-alkyl.
L is selected from a covalent bond, —CHR⁵—, —O—, —OCH₂—, —CH₂O—, —CH₂OCH₂—, —CF₂CH₂—, and —CH₂CF₂—;
A is selected from C₆-C₁₄-aryl, 5- to 14-membered heteroaryl, and 3- to 14-membered heterocyclyl;
R³ and R⁴ are independently selected from hydrogen, halogen, SF₅, cyano, C₁₋₆-alkyl, C₁₋₆-alkoxy, halo-C₁₋₆-alkyl, halo-C₁₋₆-alkoxy, C₆-C₁₄-aryl, C₃-C₁₀-cycloalkyl, 5-14-membered heteroaryl, C₆-C₁₄-aryloxy, C₃-C₁₀-cycloalkyloxy, and 5-14-membered heteroaryloxy, wherein said C₆-C₁₄-aryl, C₃-C₁₀-cycloalkyl, 5-14-membered heteroaryl, C₆-C₁₄-aryloxy, C₃-C₁₀-cycloalkyloxy, and 5-14-membered heteroaryloxy, are optionally substituted with 1-2 substituents selected from halogen, C₁₋₆-alkyl, and halo-C₁₋₆-alkyl; and
R⁵ is selected from hydrogen and C₆-C₁₄-aryl.

The invention also provides the following enumerated Embodiments (E) of the first and second aspect (A1 and A2) of the invention:
E1. The compound of formula (I) according to A1 or A2, or a pharmaceutically acceptable salt thereof, wherein:
   (i) U is CH₂;
      V is O;
      W and X are both CH;
      R¹ is selected from halogen and C₁₋₆-alkyl; and
      R² is selected from hydrogen and halogen; or
   (ii) U is CH₂;
      V is O;
      W and X together form a group C═C; and
      R¹ and R² are both hydrogen; or
   (iii) U is CH₂;
      V is selected from NH, S, and CH₂;
      (a) W and X are both CH; or
      (b) W and X together form a group C═C; and
      R¹ and R² are both hydrogen; or
   (iv) U and V together form a group C═C;
      W and X together form a group C═C; and
      R¹ and R² are both hydrogen; or
   (v) U is CH₂;
      V is O;
      W is CH;
      X is C—OH; and
      R¹ and R² are both hydrogen.
E2. The compound of formula (I) according to A1 or A2, or a pharmaceutically acceptable salt thereof, wherein:
   (i) U is CH₂;
      V is O;
      W and X are both CH;
      R¹ is selected from halogen and C₁₋₆-alkyl; and
      R² is selected from hydrogen and halogen; or
   (ii) U is CH₂;
      V is O;
      W and X together form a group C═C; and
      R¹ and R² are both hydrogen; or
   (iii) U is CH₂;
      V is selected from NH and CH₂;
      (a) W and X are both CH; or
      (b) W and X together form a group C═C; and
      R¹ and R² are both hydrogen; or
   (iv) U and V together form a group C═C;
      W and X together form a group C═C; and
      R¹ and R² are both hydrogen.
E3. The compound of formula (I) according to A1 or A2, or a pharmaceutically acceptable salt thereof, wherein
   (i) U is CH₂;
      V is O;
      W and X are both CH;
      R¹ is selected from halogen and C₁₋₆-alkyl; and
      R² is selected from hydrogen and halogen; or
   (ii) U is CH₂;
      V is NH;
      W and X are both CH; and
      R¹ and R² are both hydrogen; or
   (iii) U and V together form a group C═C;
      W and X together form a group C═C; and
      R¹ and R² are both hydrogen.
E4. The compound of formula (I) according to A1 or A2, or a pharmaceutically acceptable salt thereof, wherein
   (i) U is CH₂;
      V is O;
      W and X are both CH;
      R¹ is selected from fluoro and methyl; and
      R² is selected from hydrogen and fluoro; or
   (ii) U is CH₂;
      V is NH;
      W and X are both CH; and
      R¹ and R² are both hydrogen; or
   (iii) U and V together form a group C═C;
      W and X together form a group C═C; and
      R¹ and R² are both hydrogen.
E5. The compound of formula I according to any one of A1, A2 and E1 to E4, or a pharmaceutically acceptable salt thereof, wherein Z is N.
E6. The compound of formula I according to any one of A1, A2 and E1 to E5, or a pharmaceutically acceptable salt thereof, wherein Q is CH.
E7. The compound of formula I according to any one of A1, A2 and E1 to E6, or a pharmaceutically acceptable salt thereof, wherein m and n are both 0.
E8. The compound of formula I according to any one of A1, A2 and E1 to E7, or a pharmaceutically acceptable salt thereof, wherein L is selected from a covalent bond, —CHR⁵—, and —CH₂O—.
E9. The compound of formula I according to any one of A1, A2 and E1 to E7, or a pharmaceutically acceptable salt thereof, wherein L is selected from a covalent bond and —CH₂O—.
E10. The compound of formula I according to any one of A1, A2 and E1 to E9, or a pharmaceutically acceptable salt thereof, wherein A is C₆-C₁₄-aryl.
E11. The compound of formula I according to any one of A1, A2 and E1 to E9, or a pharmaceutically acceptable salt thereof, wherein A is phenyl.
E12. The compound of formula I according to any one of A1, A2 and E1 to E11, or a pharmaceutically acceptable salt thereof, wherein R³ is selected from hydrogen and halo-C₁-C₆-alkyl.
E13. The compound of formula I according to any one of A1, A2 and E1 to E11, or a pharmaceutically acceptable salt thereof, wherein R³ is halo-C₁-C₆-alkyl.
E14. The compound of formula I according to any one of A1, A2 and E1 to E11, or a pharmaceutically acceptable salt thereof, wherein R³ is selected from CF₃ and 2,2,2-trifluoroethyl.
E15. The compound of formula I according to any one of A1, A2 and E1 to E14, or a pharmaceutically acceptable salt thereof, wherein R⁴ is selected from hydrogen and halogen.
E16. The compound of formula I according to any one of A1, A2 and E1 to E14, or a pharmaceutically acceptable salt thereof, wherein R⁴ is selected from hydrogen and fluoro.
E17. The compound of formula (I) according to A1 or A2, or a pharmaceutically acceptable salt thereof, wherein:
   (i) U is CH₂;
      V is O;
      W and X are both CH;
      R¹ is selected from halogen and C₁₋₆-alkyl; and
      R² is selected from hydrogen and halogen; or
   (ii) U is CH₂;
      V is O;
      W and X together form a group C═C; and
      R¹ and R² are both hydrogen; or
   (iii) U is CH₂;
      V is selected from NH, S, and CH₂;
      (a) W and X are both CH; or
      (b) W and X together form a group C═C; and
      R¹ and R² are both hydrogen; or
   (iv) U and V together form a group C═C;
      W and X together form a group C═C; and
      R¹ and R² are both hydrogen; or
   (v) U is CH₂;
      V is O;
      W is CH;
      X is C—OH; and
      R¹ and R² are both hydrogen;
   Z is N;
   Q is CH;
   m and n are both 0;
   L is selected from a covalent bond, —CHR⁵—, and —CH₂O—;
   A is C₆-C₁₄-aryl;
   R³ is selected from hydrogen and halo-C₁-C₆-alkyl;
   R⁴ is selected from hydrogen and halogen; and
   R⁵ is selected from hydrogen and C₆-C₁₄-aryl.
E18. The compound of formula (I) according to A1 or A2, or a pharmaceutically acceptable salt thereof, wherein:
   (i) U is CH₂;
      V is O;
      W and X are both CH;
      R¹ is selected from halogen and C₁₋₆-alkyl; and
      R² is selected from hydrogen and halogen; or
   (ii) U is CH₂;
      V is O;
      W and X together form a group C═C; and
      R¹ and R² are both hydrogen; or
   (iii) U is CH₂;
      V is selected from NH and CH₂;
      (a) W and X are both CH; or
      (b) W and X together form a group C═C; and
      R¹ and R² are both hydrogen; or
   (iv) U and V together form a group C═C;
      W and X together form a group C═C; and
      R¹ and R² are both hydrogen;
   Z is N;
   Q is CH;
   m and n are both 0;
   L is selected from a covalent bond, —CHR⁵—, and —CH₂O—;
   A is C₆-C₁₄-aryl;
   R³ is selected from hydrogen and halo-C₁-C₆-alkyl;
   R⁴ is selected from hydrogen and halogen; and
   R⁵ is selected from hydrogen and C₆-C₁₄-aryl.
E19. The compound of formula (I) according to A1 or A2, or a pharmaceutically acceptable salt thereof, wherein
   (i) U is CH₂;
      V is O;
      W and X are both CH;
      R¹ is selected from halogen and C₁₋₆-alkyl; and
      R² is selected from hydrogen and halogen; or
   (ii) U is CH₂;
      V is NH;
      W and X are both CH; and
      R¹ and R² are both hydrogen; or
   (iii) U and V together form a group C═C;
      W and X together form a group C═C; and
      R¹ and R² are both hydrogen;
   Z is N;
   Q is CH;
   m and n are both 0;
   L is selected from a covalent bond and —CH₂O—;
   A is C₆-C₁₄-aryl;
   R³ is halo-C₁-C₆-alkyl; and
   R⁴ is selected from hydrogen and halogen.
E20. The compound of formula (I) according to A1 or A2, or a pharmaceutically acceptable salt thereof, wherein
   (i) U is CH₂;
      V is O;
      W and X are both CH;
      R¹ is selected from fluoro and methyl; and
      R² is selected from hydrogen and fluoro; or
   (ii) U is CH₂;
      V is NH;
      W and X are both CH; and
      R¹ and R² are both hydrogen; or
   (iii) U and V together form a group C═C;
      W and X together form a group C═C; and
      R¹ and R² are both hydrogen;
   Z is N;
   Q is CH;
   m and n are both 0;
   L is selected from a covalent bond and —CH₂O—;
   A is phenyl;
   R³ is selected from CF₃ and 2,2,2-trifluoroethyl; and
   R⁴ is selected from hydrogen and fluoro.
E21. The compound of formula (I) according to any one of A1, A2 and E1 to E20, or a pharmaceutically acceptable salt thereof, selected from:
rel-(4aR,8S,8aS)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one;
rel-(4aS,8R,8aR)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one;
rel-(4aS,8aS)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one;
rel-(4aR,8aR)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one;
rel-(4aS,8aS)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one;
rel-(4aR,8aR)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one;
6-[4-[[4-(Trifluoromethyl)phenyl]methyl]piperidine-1-carbonyl]-4,5,7,8-tetrahydropyrido[4,3-b][1,4]oxazin-3-one;
7-(4-Benzhydrylpiperidine-1-carbonyl)-1,5,6,8-tetrahydro-1,7-naphthyridin-2-one;
7-[3-[[2-Fluoro-4-(trifluoromethyl)phenyl]methoxy]azetidine-1-carbonyl]-1,5,6,8-tetrahydro-1,7-naphthyridin-2-one;
rac-(4aS,8aS)-7-(4-Benzhydrylpiperidine-1-carbonyl)-1,3,4,4a,5,6,8,8a-octahydro-1,7-naphthyridin-2-one;
rac-(4aS,8a5)-7-[3-[[2-Fluoro-4-(trifluoromethyl)phenyl]methoxy]azetidine-1-carbonyl]-1,3,4,4a,5,6,8,8a-octahydro-1,7-naphthyridin-2-one;
rac-(4aR,8aS)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one;
(4aR,8aS)- or (4a5,8aR)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one;
(4aS,8aR)- or (4aR,8aS)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one;
6-[3-[[2-Fluoro-4-(trifluoromethyl)phenyl]methoxy]azetidine-1-carbonyl]-1,2,4,5,7,8-hexahydropyrido[3,4-b]pyrazin-3-one;
(4a5,8a5)-6-[3-[[2-fluoro-4-(trifluoromethyl)phenyl]methoxy]azetidine-1-carbonyl]-4a-hydroxy-5,7,8,8a-tetrahydro-4H-pyrido[4,3-b][1,4]oxazin-3-one;
rac-(4aS,8aS)-7-[3-[[2-fluoro-4-(trifluoromethyl)phenyl]methoxy]azetidine-1-carbonyl]-4-hydroxy-1,3,4,4a,5,6,8,8a-octahydro-1,7-naphthyridin-2-one; and
6-[3-[[2-fluoro-4-(trifluoromethyl)phenyl]methoxy]azetidine-1-carbonyl]-4,5,7,8-tetrahydropyrido[4,3-b][1,4]thiazin-3-one.
E22. The compound of formula (I) according to any one of A1, A2 and E1 to E20, or a pharmaceutically acceptable salt thereof, selected from:
(4aS,8aS)- or (4aR,8aR)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one;
rac-(4aR,8aR)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one;
(4aR,8S,8aS)- or (4aR,8R,8aS)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one;
7-[3-[[2-Fluoro-4-(trifluoromethyl)phenyl]methoxy]azetidine-1-carbonyl]-1,5,6,8-tetrahydro-1,7-naphthyridin-2-one; and
rac-(4aR,8aS)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one.
E23. The compound of formula (I) according to A1 or A2, or a pharmaceutically acceptable salt thereof, wherein:
   L is selected from a covalent bond, —CHR⁵—, and —CH₂O—;
   A is C₆-C₁₄-aryl;
   R³ is selected from hydrogen and halo-C₁-C₆-alkyl;
   R⁴ is selected from hydrogen and halogen; and
   R⁵ is selected from hydrogen and C₆-C₁₄-aryl.
E24. The compound of formula (I) according to A1 or A2, or a pharmaceutically acceptable salt thereof, wherein
   L is selected from a covalent bond and —CH₂O—;
   A is C₆-C₁₄-aryl;
   R³ is halo-C₁-C₆-alkyl; and
   R⁴ is selected from hydrogen and halogen.
E25. The compound of formula (I) according to A1 or A2, or a pharmaceutically acceptable salt thereof, wherein
   L is selected from a covalent bond and —CH₂O—;
   A is phenyl;
   R³ is selected from CF₃ and 2,2,2-trifluoroethyl; and
   R⁴ is selected from hydrogen and fluoro.
A3. In a further aspect, the present invention provides a compound of formula (I) according to A1 or A2, or a pharmaceutically acceptable salt thereof, wherein the compound of formula (I) is a compound of formula (II):

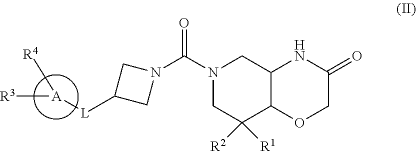

wherein
A is C₆-C₁₄-aryl;
L is a covalent bond or CH₂O;
R¹ is selected from halogen and C₁₋₆-alkyl;
R² is selected from hydrogen and halogen;
R³ is halo-C₁₋₆-alkyl; and
R⁴ is selected from hydrogen and halogen.
E26. The compound of formula (II) according to A3, or a pharmaceutically acceptable salt thereof, wherein:
   A is phenyl;
   L is a covalent bond or CH₂O;
   R¹ is selected from fluoro and methyl;
   R² is selected from hydrogen and fluoro;
   R³ is selected from CF₃ and 2,2,2-trifluoroethyl; and
   R⁴ is selected from hydrogen and fluoro.
A4. In a further aspect, the present invention provides a compound of formula (I) according to A1 or A2, or a pharmaceutically acceptable salt thereof, wherein the compound of formula (I) is a compound of formula (III):

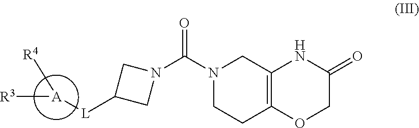

wherein:
A is C₆-C₁₄-aryl; and
R³ and R⁴ are independently selected from hydrogen and halo-C₁₋₆-alkyl.
E27. The compound of formula (III) according to A4, or a pharmaceutically acceptable salt thereof, wherein:
   A is C₆-C₁₄-aryl;
   R³ is halo-C₁₋₆-alkyl; and
   R⁴ is hydrogen.
E28. The compound of formula (III) according to A4, or a pharmaceutically acceptable salt thereof, wherein:
   A is phenyl;
   R³ is CF₃; and
   R⁴ is hydrogen.
A5. In a further aspect, the present invention provides a compound of formula (I) according to A1 or A2, or a pharmaceutically acceptable salt thereof, wherein the compound of formula (I) is a compound of formula (IV):

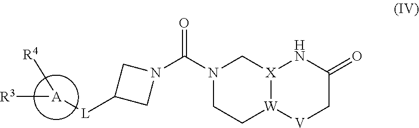

wherein:
V is selected from NH, S, and CH₂;
(a) W and X are both CH; or
(b) W and X together form a group C═C;
A is C₆-C₁₄-aryl;
L is selected from —CHR⁵— and —CH₂O—;
R³ is selected from hydrogen and halo-C₁₋₆-alkyl;
R⁴ is selected from hydrogen and halogen; and
R⁵ is C₆-C₁₄-aryl.
E29. The compound of formula (IV) according to A5, or a pharmaceutically acceptable salt thereof, wherein:
   V is selected from NH and CH₂;
   (c) W and X are both CH; or
   (d) W and X together form a group C═C;
   A is C₆-C₁₄-aryl;
   L is selected from —CHR⁵— and —CH₂O—;
   R³ is selected from hydrogen and halo-C₁₋₆-alkyl;
   R⁴ is selected from hydrogen and halogen; and
   R⁵ is C₆-C₁₄-aryl.
E30. The compound of formula (IV) according to A5, or a pharmaceutically acceptable salt thereof, wherein:
V is NH;
W and X are both CH;
A is C₆-C₁₄-aryl;
L is —CH₂O—;
R³ is halo-C₁₋₆-alkyl; and
R⁴ is halogen.
E31. The compound of formula (IV) according to A5, or a pharmaceutically acceptable salt thereof, wherein:
   V is NH;
   W and X are both CH;
   A is phenyl;
   L is —CH₂O—;
   R³ is CF₃; and
   R⁴ is fluoro.
A6. In a further aspect, the present invention provides a compound of formula (I) according to A1 or A2, or a pharmaceutically acceptable salt thereof, wherein the compound of formula (I) is a compound of formula (V):

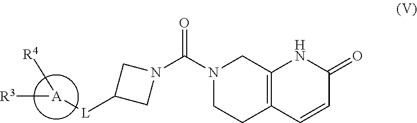

wherein:
A is C₆-C₁₄-aryl;
L is selected from —CHR⁵— and —CH₂O—;
R³ is selected from hydrogen and halo-C₁₋₆-alkyl;
R⁴ is selected from hydrogen and halogen; and
R⁵ is C₆-C₁₄-aryl.
E32. The compound of formula (V) according to A6, or a pharmaceutically acceptable salt thereof, wherein:
A is C₆-C₁₄-aryl;
L is —CH₂O—;
R³ is halo-C₁₋₆-alkyl; and
R⁴ is halogen.
E33. The compound of formula (V) according to A6, or a pharmaceutically acceptable salt thereof, wherein:
   A is phenyl;
   L is —CH₂O—;
   R³ is CF₃; and
   R⁴ is fluoro.

In a particular embodiment, the present invention provides pharmaceutically acceptable salts of the compounds according to formula (I) as described herein, especially hydrochloride salts. In a further particular embodiment, the present invention provides compounds according to formula (I) as described herein as free bases.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:

AcOH=acetic acid, ACN=acetonitrile, Bn=benzyl, Boc=tert-butyloxycarbonyl, CAS RN=chemical abstracts registration number, Cbz=benzyloxycarbonyl, CPME=cyclopentyl methyl ether, Cs₂CO₃=cesium carbonate, CO=carbon monoxide, CuCl=copper(I) chloride, CuCN=copper(I) cyanide, CuI=copper(I) iodide, DAST=(diethylamino)sulfur trifluoride, DBU=1,8-diazabicyclo[5,4,0]undec-7-ene, DEAD=diethyl azodicarboxylate, DIAD=diisopropyl azodicarboxylate, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC.HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluorophosphate, HCl=hydrogen chloride, HOBt=1-hydroxy-1H-benzotriazole; HPLC=high performance liquid chromatography, iPrMgCl=isopropylmagnesium chloride, I₂=iodine, IPA=2-propanol, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, mCPBA=meta-chloroperoxybenzoic acid, MgSO₄=magnesium sulfate, min=minute(s), mL=milliliter, MPLC=medium pressure liquid chromatography, MS=mass spectrum, nBuLi=n-butyllithium, NaBH₃CN=sodium cyanoborohydride, NaH=sodium hydride, NaHCO₃=sodium hydrogen carbonate, NaNO₂=sodium nitrite, NaBH(OAc)₃=sodium triacetoxyborohydride, NaOH=sodium hydroxide, Na₂CO₃=sodium carbonate, Na₂SO₄=sodium sulfate, Na₂S₂O₃=sodium thiosulfate, NBS=N-bromosuccinimide, nBuLi=n-butyllithium, NEt₃=triethylamine (TEA), NH₄Cl=ammonium chloride, NMP=N-methyl-2-pyrrolidone, OAc=Acetoxy, T₃P=propylphosphonic anhydride, PE=petroleum ether, PG=protective group, Pd—C=palladium on activated carbon, PdCl₂(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(0), Pd(OAc)₂=palladium(II) acetate, Pd(OH)₂=palladium hydroxide, Pd(PPh₃)₄=tetrakis(triphenylphosphine)palladium(0), PTSA=p-toluenesulfonic acid, R=any group, RT=room temperature, SFC=Supercritical Fluid Chromatography, S-PHOS=2-dicyclohexylphosphino-2′,6′-dimethoxybiphenyl, TBAI=tetra butyl ammonium iodine, TBME=tert-butyl methyl ether, TEA=triethylamine, TFA=trifluoracetic acid, THF=tetrahydrofuran, TMEDA=N,N,N′,N′-tetramethylethylenediamine, ZnCl₂=zinc chloride, Hal=halogen.

Compounds of formula I wherein U, V, W, X, Q, L, A, m, n, and R¹ to R⁴ are as described herein can be synthesized in analogy to literature procedures and/or as depicted for example in Scheme 1a.

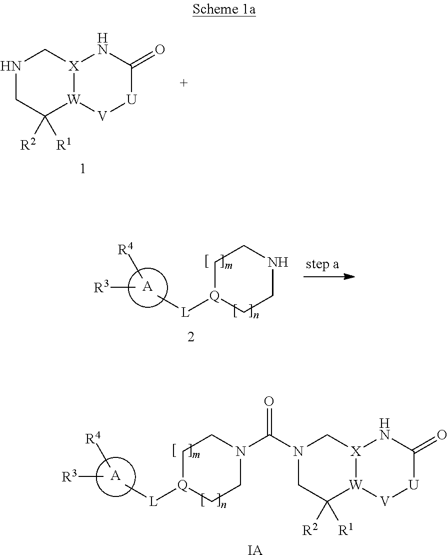

Accordingly, bicyclic piperazines 1 are reacted with intermediates 2 in the presence of an urea forming reagent such as bis(trichloromethyl) carbonate using a suitable base and solvent such as, e.g. sodium bicarbonate in DCM, to give compounds of formula IA (step a). Further urea forming reagents include but are not limited to phosgene, trichloromethyl chloroformate, (4-nitrophenyl)carbonate or 1,1′-carbonyldiimidazole. Reactions of this type and the use of these reagents are widely described in literature (e.g. G. Sartori et al., Green Chemistry 2000, 2, 140). A person skilled in the art will acknowledge that the order of the addition of the reagents can be important in this type of reactions due to the reactivity and stability of the intermediary formed carbamoyl chlorides, as well as for avoiding formation of undesired symmetrical urea by-products.

Compounds of formula IB wherein U, V, W, X, Q, L, A, m, n, and R¹ to R⁴ are as described herein can be synthesized in analogy to literature procedures and/or as depicted for example in Scheme 1b.

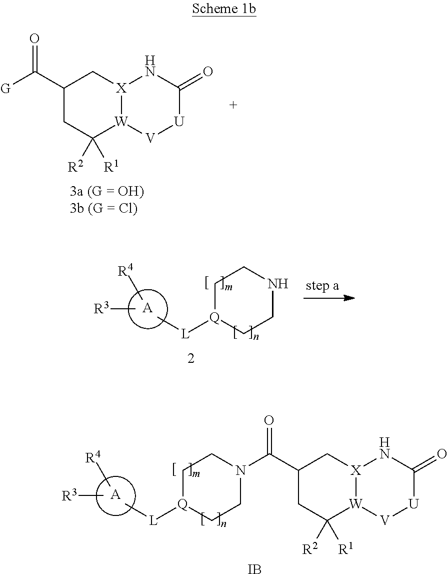

Accordingly, intermediates 2 can be coupled with an activated form of a bicyclic carboxylic acid 3a (G=OH) or alternatively with carboxylic acid chlorides 3b (G=Cl) to provide compounds IB (step a). Amide couplings of this type are widely described in the literature and can be accomplished by the usage of coupling reagents such as CDI, DCC, HATU, HBTU, HOBT, TBTU, T3P or Mukaiyama reagent (Mukaiyama T. Angew. Chem., Int. Ed. Engl. 1979, 18, 707) in a suitable solvent e.g., DMF, DMA, DCM or dioxane, optionally in the presence of a base (e.g., TEA, DIPEA (Huenig's base) or DMAP).

Alternatively, the carboxylic acids 3a can be converted into their acid chlorides 3b by treatment with, e.g. thionyl chloride or oxalyl chloride, neat or optionally in a solvent such as DCM. Reaction of the acid chloride with intermediates 2 in an appropriate solvent such as DCM or DMF and a base, e.g. TEA, Huenig's base, pyridine, DMAP or lithium bis(trimethylsilyl)amide at temperatures ranging from 0° C. to the reflux temperature of the solvent or solvent mixture yields compounds IB (step a).

In some embodiments bicyclic piperazine intermediates 1 are intermediates of type 1a. Intermediates of type 1a in which R² is C₁₋₆ alkyl can be prepared by methods well known by a person skilled in the art and as exemplified by the general synthetic procedure outlined in Scheme 2.

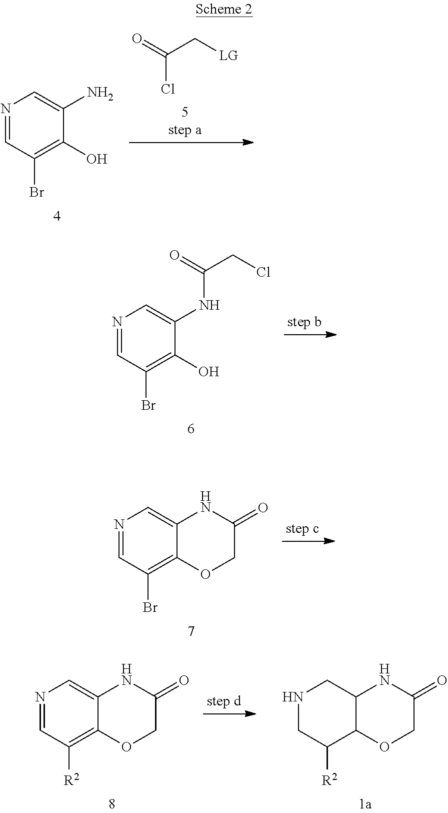

Commercially available 3-amino-5-bromo-pyridin-4-ol 4 can be acylated for example with chloro- or bromoacetyl chloride 5, in which “LG” signifies a suitable leaving group (e.g., Cl or Br), using a suitable base such as sodium or potassium carbonate, sodium hydroxide or sodium acetate in an appropriate solvent such as THF, water, acetone or mixtures thereof, to provide intermediates 6 (step a).

Intermediates 6 can be cyclized to intermediates 7 using methods well known in the art, for example by treatment of 6 with sodium hydride in THF or potassium tert-butoxide in IPA and water (step b). Reactions of that type are described in literature (e.g., Z. Rafinski et al., J. Org. Chem. 2015, 80, 7468; S. Dugar et al., Synthesis 2015, 47(5), 712; WO2005/066187).

The bromine in intermediates 7 can exchanged for example to a C₁₋₆-alkyl group by reacting intermediates 7 with C₁₋₆-alkyl boronic acids of type R²B(OH)₂ or boronic esters of type R²B(OR)₂ (e.g. 4,4,5,5-tetramethyl-2-phenyl-1,3,2-dioxaborolane (pinacol) ester), either commercially available or prepared using literature procedures as described for example in “Boronic Acids—Preparation and Applications in Organic Synthesis and Medicine” by Dennis G. Hall (ed.) 1st Ed., 2005, John Wiley & Sons, New York) using a suitable catalyst (e.g. dichloro[1,1′-bis(diphenylphosphino)-ferrocene]palladium(II) dichloromethane adduct, tetrakis(triphenylphosphine)palladium(0) or palladium(II)acetate with triphenylphosphine) in an appropriate solvent (e.g. dioxane, dimethoxyethane, water, toluene, DMF or mixtures thereof) and a suitable base (e.g. Na₂CO₃, NaHCO₃, KF, K₂CO₃ or TEA) at temperatures between room temperature and the boiling point of the solvent or solvent mixture, to yield intermediates 8 (step c). Suzuki reactions of this type are broadly described in literature (e.g. A. Suzuki, Pure Appl. Chem. 1991, 63, 419-422; A. Suzuki, N. Miyaura, Chem. Rev. 1995, 95, 2457-2483; A. Suzuki, J. Organomet. Chem. 1999, 576, 147-168; V. Polshettiwar et al., Chem. Sus. Chem. 2010, 3, 502-522) and are well known to those skilled in the art.

Intermediates 8 can be reduced to bicyclic piperazines 1a for example applying heterogeneous catalytic hydrogenation using a catalyst such as Pd(OH)₂, Pd/C or Rh/C in a solvent like THF, MeOH, EtOH, EtOAc or a mixture thereof, optionally in the presence of acid such as sulfuric acid at temperatures ranging from RT to the boiling point of the solvent at atmospheric or elevated pressure of hydrogen (step d).

In some embodiments bicyclic piperazine intermediates 1 are intermediates of type 1b. Intermediates of type 1b in which R¹═R²═F can be prepared by methods well known by a person skilled in the art and as exemplified by the general synthetic procedure outlined in Scheme 3.

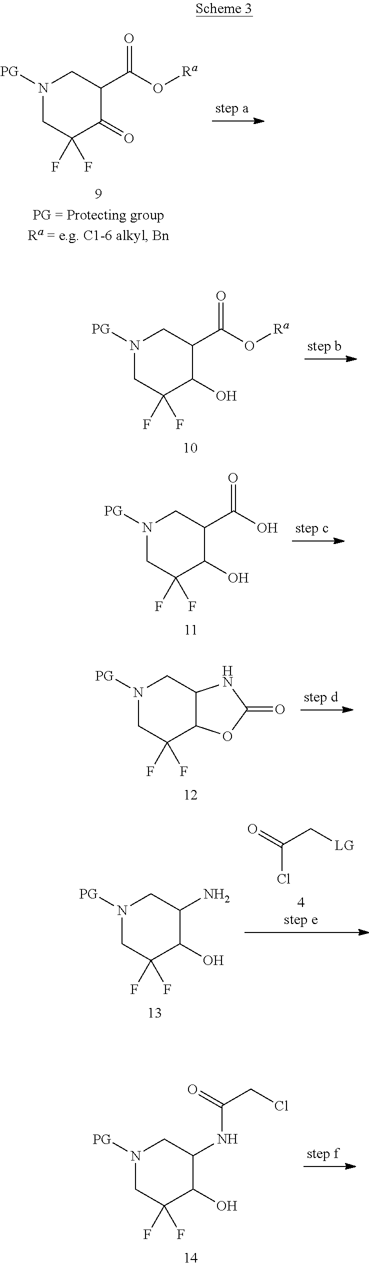

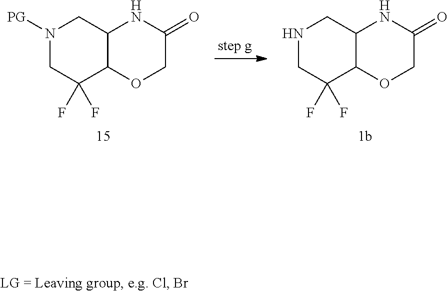

The ketone in commercially available 5,5-difluoro-4-oxopiperidines of type 9 in which PG is a suitable protecting group such as a Boc protecting group and R^{a} is for example methyl can be reduced to the alcohol function for example by using sodium or potassium borohydride in a suitable solvent such as MeOH or EtOH at temperatures ranging from 0° C. to the boiling point of the solvent to provide intermediates 10 (step a). Alternatively, the ketone functionality can be reduced by enzymatic means as known in the art and published in literature (e.g. Acc. Chem. Res. 2007, 40, 12, 1412-1419) (step a).

Cleavage of the ester group in intermediates 10 using methods well known in the art, for example a methyl ester by reaction with a base such as LiOH or NaOH in a solvent such as MeOH, EtOH, THF or mixtures thereof, yields intermediates 11 (step b).

The carboxylic acid functionality in intermediates 11 can be reacted with an azide source such as diphenylphosphoryl azide in the presence of a base such as, e.g. TEA in a solvent such as toluene at elevated temperatures up to the boiling point of the solvent. Subsequent intramolecular addition of the alcohol group onto the isocyanate from the intermediary formed acylazide provides intermediates 12 (step c).

Opening of the oxazolidinone ring of intermediates 12 using a base such as sodium hydroxide in a suitable solvent such as cyclopentyl methyl ether at elevated temperatures yields intermediates 13 (step d).

Intermediates 13 can be acylated for example with chloro- or bromoacetyl chloride 4 for example applying the conditions described under Scheme 2, step a), to provide intermediates 14 (step e).

Intermediates 14 can be cyclized for example using the conditions described under Scheme 2, step b), to furnish intermediates 15 (step f).

Removal of the protecting groups from intermediates 15 using conditions well known in the art, e.g., a Boc group using TFA in DCM or HCl in EtOH or EtOAc at temperatures between 0° C. and room temperature yields intermediates 1b (step g).

In some embodiments bicyclic piperazine intermediates 1 are intermediates of type 1c. Intermediates of type 1c can be prepared by methods well known by a person skilled in the art and as exemplified by the general synthetic procedure outlined in Scheme 4.

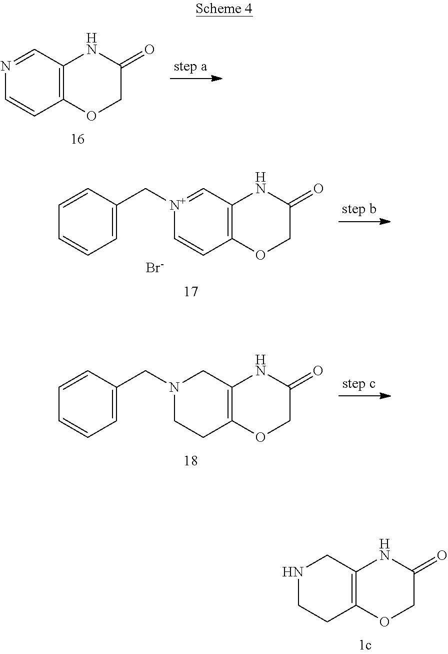 [SMILES: O=C1COc2ccncc2N1 ; O=C1COc2cc[n+](Cc3ccccc3)cc2N1 ; [Br-] ; O=C1COC2=C(CN(Cc3ccccc3)CC2)N1 ; O=C1COC2=C(CNCC2)N1]

Commercially available 2H-pyrido[4,3-b][1,4]oxazin-3(4h)-one 16 can be reacted with benzyl bromide in a suitable solvent such as methanol to give intermediate 17 (step a).

Reduction of intermediate 17 for example with sodium borohydride in an appropriate solvent such as EtOH provides intermediates 18 (step b).

Removal of the benzyl group in intermediates 18 by methods know in the art, for example by hydrogenation using a suitable catalyst and solvent such as Pd/C in MeOH, optionally in the presence of acetyl chloride furnishes intermediates 1c (step c).

In some embodiments bicyclic piperazine intermediates 1 are intermediates of type 1d. Intermediates of type 1d can be prepared by methods well known by a person skilled in the art and as exemplified by the general synthetic procedure outlined in Scheme 5.

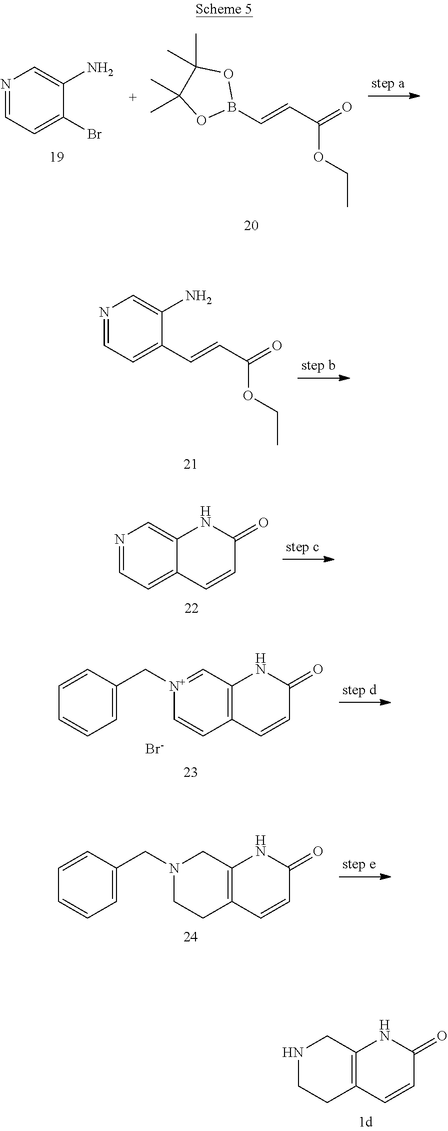

Commercially available 4-bromopyridin-3-amine 19 can be reacted with boronic acid ester 20, either commercially available or prepared by methods known in the art, in the presence of a suitable catalyst and base such as 1,1-bis(di-tert-butylphosphino)ferrocene palladium dichloride and K₂CO₃ in an appropriate solvent such as DMF at temperatures ranging from RT to the boiling point of the solvent to provide intermediates 21 (step a).

Intermediates 21 can be reacted for example with a suitable base such as sodium methanolate in a suitable solvent such as MeOH followed by reaction with hydroxylamine hydrochloride and subsequent heating to yield intermediates 22 (step b).

Intermediates 22 can be transformed into intermediates 23 using for example the conditions described under Scheme 4, step a (step c).

Intermediates 23 can be further converted into intermediates 24 applying for example the conditions described under Scheme 4, step b (step d).

Removal of the benzyl group from intermediates 24 using for example the conditions described under Scheme 4, step c, yields compounds 1d (step e).

In some embodiments bicyclic piperazine intermediates 1 are intermediates of type 1e. Intermediates of type 1e can be prepared by methods well known by a person skilled in the art and as exemplified by the general synthetic procedure outlined in Scheme 6.

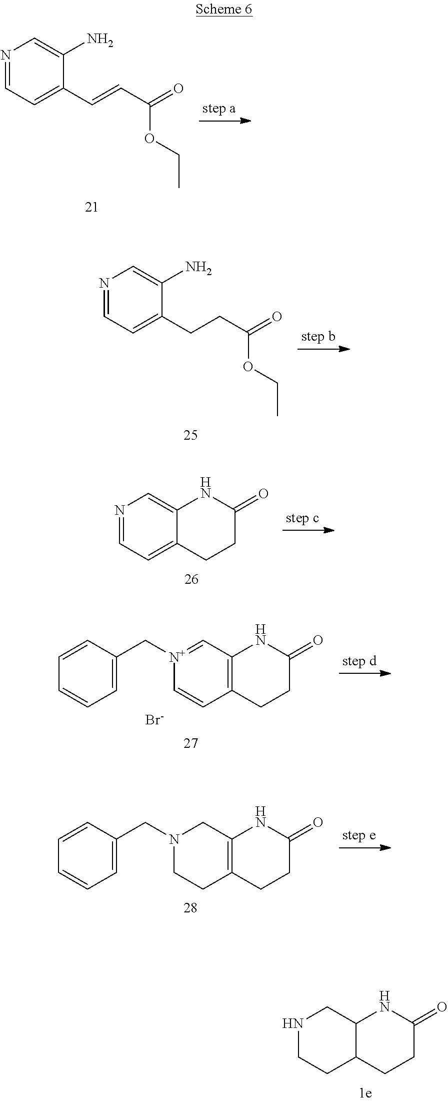

The double bond in intermediates 21 (prepared according to Scheme 5, step a) can be reduced by methods known in the art and for example by hydrogenation using a suitable catalyst and solvent such as Pd/C in MeOH to provide intermediates 25 (step a).

Intermediates 25 can be cyclized to intermediates 26 for example under acidic conditions using a mixture of AcOH and HCl, optionally at elevated temperatures (step b).

Intermediates 26 can be converted to intermediates 27 for example using the conditions described under Scheme 4, step a (step c).

Reduction of intermediates 27 applying the conditions described under Scheme 4, step b, furnishes intermediates 28 (step d).

Concomitant removal of the benzyl group and reduction of the bridge double bond in intermediates 28 applying for example the conditions described under Scheme 4, step c, gives intermediates 1e (step e).

In some embodiments bicyclic piperazine intermediates 1 are intermediates of type 1f and 1g. Intermediates of type 1f and 1g can be prepared by methods well known by a person skilled in the art and as exemplified by the general synthetic procedure outlined in Scheme 7.

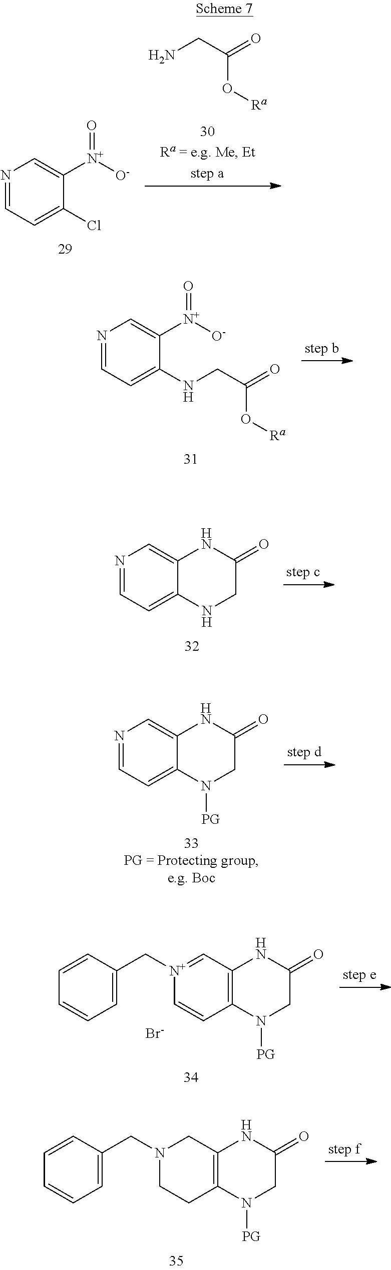

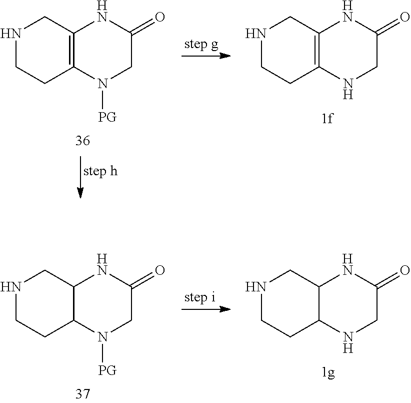

Commercially available 4-chloro-3-nitro-pyridine 29 can be reacted for example with glycine methyl ester hydrochloride (30, HCl salt, R^{a}=Me) in the presence of a suitable base such as TEA in an appropriate solvent, for example 1,4-dioxane to provide intermediates 31 (step a).

Reduction of the nitro group in intermediates 31 for example using hydrogenation in the presence of a suitable catalyst such as Pd/C in a suitable solvent like MeOH under in situ ring closure of the resulting amine onto the ester functionality gives intermediates 32 (step b).

Protection of the secondary basic nitrogen of intermediates 32 with a suitable protecting group such as a Boc group applying methods well known in the art, for example by reaction with di-tert-butyl dicarbonate using a suitable base and solvent, e.g. TEA and DMAP in DMF, furnishes intermediates 33 (step c).

Intermediates 33 can be benzylated at the pyridine nitrogen for example using the conditions described under Scheme 4, step a, to provide intermediates 34 (step d).

Intermediates 34 can be reduced for example using the conditions described under Scheme 4, step a, to give intermediates 35 (step e).

Removal of the benzyl group from intermediates 35 applying for example the conditions outlined under Scheme 4, step c, furnishes intermediates 36 (step f).

Removal of the protective group from intermediates 36 using methods well known in the art, for example a Boc using the conditions described under scheme 3, step g, provides intermediates 1f (step g).

The double bond in intermediates 36 can be reduced for example using the conditions described under scheme 6, step e, to give intermediates 37 (step h).

Removal of the protecting group in intermediates 37 by literature methods or applying the conditions described for intermediates 36 yields intermediates 1g (step i).

A person skilled in the art will acknowledge that the sequence of reaction steps f and g as well as h and i may be inverted depending on the used protecting groups.

In some embodiments compounds I are compounds of type IC and ID. Compounds of type IC and ID in which Q, L, A, m, n, R³ and R⁴ are as defined herein can be prepared by methods well known by a person skilled in the art and as exemplified by the general synthetic procedure outlined in Scheme 8.

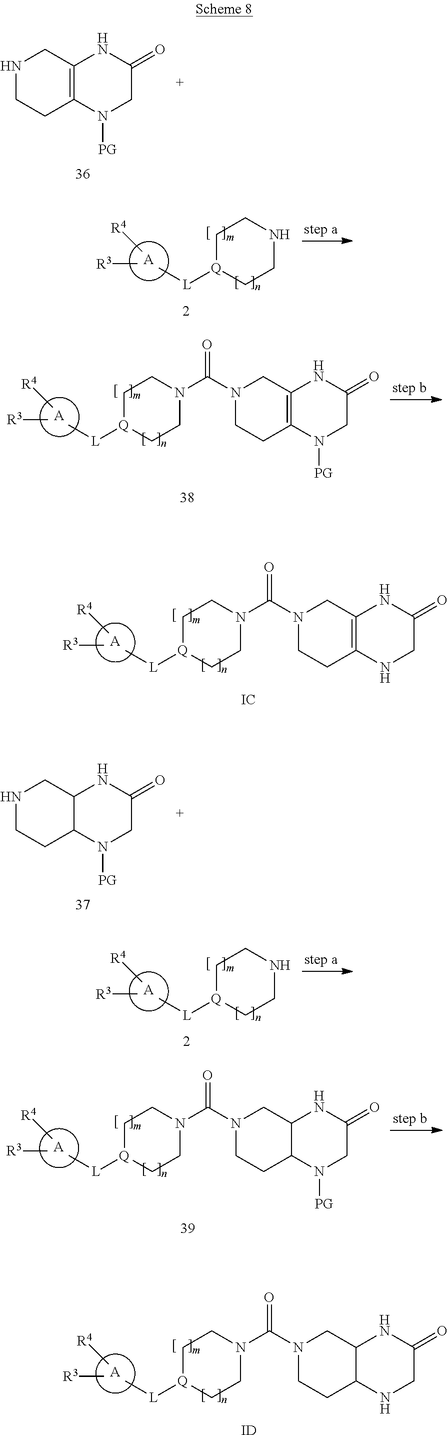

Intermediates 36 (prepared as described under scheme 7, step f) can be coupled with intermediates 2 using methods known in the art and as described under scheme 1, to give intermediates 38 (step a).

Removal of the protecting group from intermediates 38 using for example the conditions described under scheme 3, step g, furnishes compounds IC (step b).

Intermediates 37 (prepared as described under scheme 7, step h) can be coupled with intermediates 2 using methods known in the art and as described under scheme 1, to give intermediates 39 (step a).

Removal of the protecting group from intermediates 39 using for example the conditions described under scheme 3, step g, furnishes compounds ID (step b).

In some embodiments, intermediates 2 are intermediates of type 2a. Intermediates 2a in which R^{s}, m, n, A, R³ and R⁴ are as described herein and R^{q′} is hydrogen, halogen, halo-C₁₋₆-alkyl, or C₁₋₆-alkyl can be prepared by methods well known in the art and as exemplified by the general synthetic procedure outlined in Scheme 9.

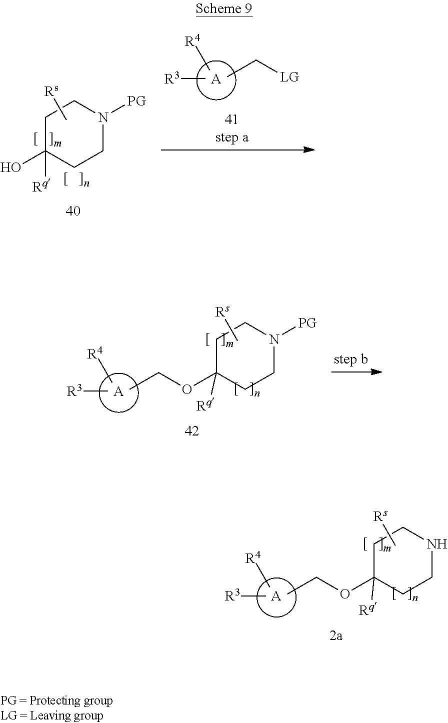

Intermediates 42 may be prepared from alcohols 40, either commercially available or prepared by methods known by a person skilled in the art and in which PG is a suitable protective group such as a Cbz, Boc or Bn, by alkylation with compounds 41 in which LG is a suitable leaving group such as chlorine, bromine, iodine, OSO₂alkyl (e.g. methanesulfonate), OSO₂fluoroalkyl (e.g. trifluoromethanesulfonate) or OSO₂aryl (e.g. p-toluenesulfonate) using a suitable base, such as sodium hydride, potassium tert-butoxide, in an appropriate solvent (e.g. in DMF or THF) at temperatures between 0° C. and the boiling temperature of the solvent (step a).

Removal of the protective group from intermediates 42 applying methods known in the art (e.g., a Boc group using TFA in DCM at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures therefore and as described for example in “Protective Groups in Organic Chemistry” by T. W. Greene and P. G. M. Wuts, 4th Ed., 2006, Wiley N.Y.), furnishes intermediates 2a (step b).

In some embodiments, intermediates 2 are intermediates of type 2b. Intermediates 2b in which R^{s}, m, n, R⁵ and A are as defined herein and R^{q} is hydrogen can be prepared by a variety of conditions, which may be exemplified by the general synthetic procedure outlined in Scheme 10.

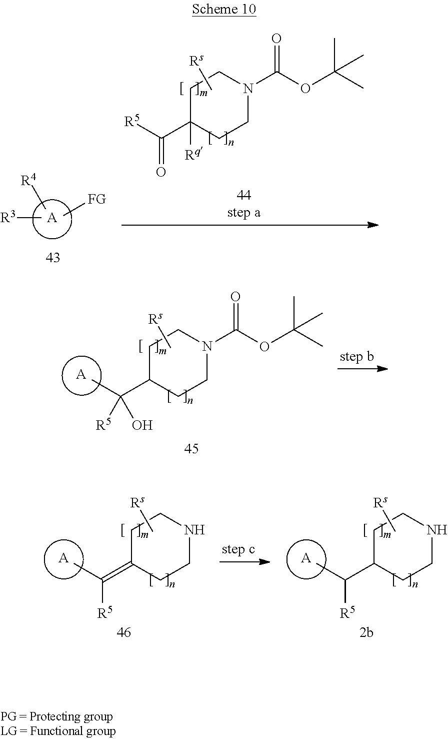

Starting from aryl or heteroaryl halides 43, wherein FG is selected from Cl, Br or I, a lithium halogen exchange reaction can be performed using a solution of LiHMDS or n-BuLi, preferably n-BuLi in a solvent like THF, diethyl ether, n-pentane, n-hexane or mixtures thereof, preferably THF and in a temperature range between −20° C. and −78° C., preferably at −78° C., to generate the corresponding lithiated aryl or heteroaryl intermediate. Nucleophilic addition of the in situ prepared lithiated aryl or heteroaryl intermediate to ketones of type 44 in which PG is a suitable protecting group such as a Boc group in a solvent such as THF and preferably at a temperature of −78° C. gives the corresponding tertiary alcohols 45 (step a).

Subsequent elimination of the tertiary hydroxy group with concomitant removal of the Boc protective group using acidic conditions such as 4M HCl in dioxane in a solvent like MeOH, or, preferably, TFA in DCM, yields the corresponding olefinic intermediates 46 (step b).

Heterogeneous catalytic hydrogenation of olefins 46 using a catalyst such as Pd(OH)₂ or Pd/C in a solvent like THF, MeOH, EtOH, EtOAc or a mixture thereof, preferably Pd/C in THF under e.g., atmospheric pressure of hydrogen, affords intermediates of type 2b (step c).

Intermediates 44 are commercially available and/or can be prepared in analogy to methods described in literature, e.g. Bioorg. Med. Chem. Lett. 2011, 21(18), 5191, WO2012/155199, WO2016/180536, Bioorg. Med. Chem. Lett. 2008, 18(18), 5087, WO2007/117557, J. Am. Chem. Soc. 2017, 139(33), 11353, J. Med. Chem. 2017, 60(13), 5507.

In some embodiments, intermediates 2 are intermediates of type 2c. Intermediates 2c in which R^{s}, m, n, R³, R⁴, and A are as described herein can be prepared by a methods known in the art and as exemplified by the general synthetic procedure outlined in Scheme 11.

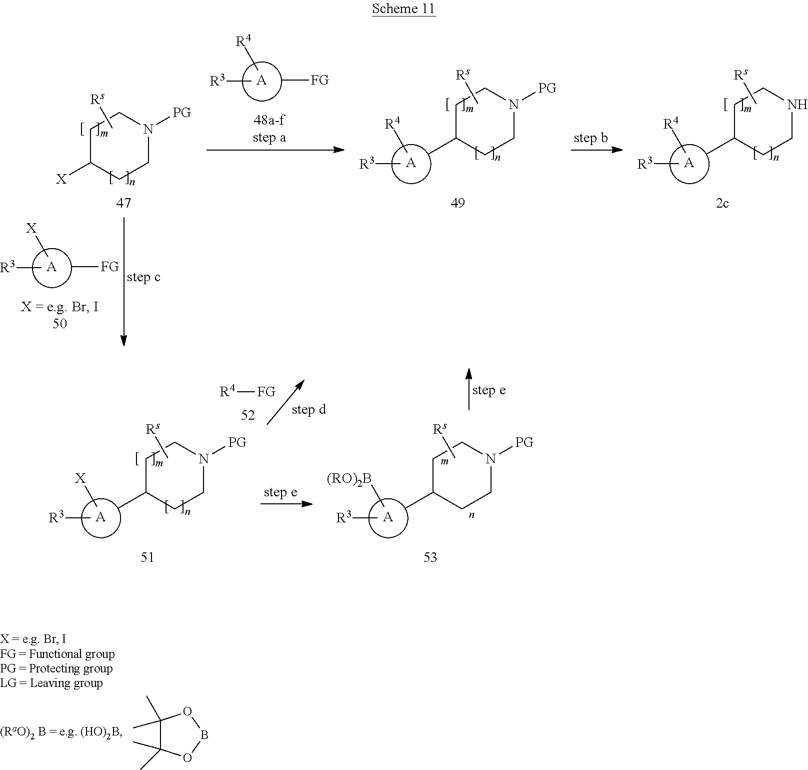

Intermediates 47 either commercially available or prepared by methods known in the art in which PG signifies a suitable protecting group and X is bromide or iodide can be subjected to cross-coupling reactions such as Negishi, Heck, Stille, Suzuki, Sonogashira or Buchwald-Hartwig coupling reactions with compounds 48, either commercially available or prepared by methods known in the art, in which FG signifies a suitable functional group such as, e.g. chloro, bromo, iodo, —OSO₂alkyl (e.g. mesylate (methanesulfonate), —OSO₂fluoroalkyl (e.g. triflate (trifluoromethanesulfonate) or —OSO₂aryl (e.g. tosylate (p-toluenesulfonate).

Reactions of this type are broadly described in literature and well known to persons skilled in the art (step a).

For example, intermediates 47 can be reacted with aryl or heteroaryl boronic acids 48a (FG=B(OH)₂) or boronic esters 48b (FG=e.g. 4,4,5,5-tetramethyl-2-phenyl-1,3,2-dioxaborolane (pinacol) ester) either commercially available or prepared using literature procedures as described for example in “Boronic Acids—Preparation and Applications in Organic Synthesis and Medicine” by Dennis G. Hall (ed.) 1st Ed., 2005, John Wiley & Sons, New York) using a suitable catalyst (e.g. dichloro[1,1′-bis(diphenylphosphino)-ferrocene]palladium(II) dichloromethane adduct, tetrakis(triphenylphosphine)palladium(0) or palladium(II)acetate with triphenylphosphine) in an appropriate solvent (e.g. dioxane, dimethoxyethane, water, toluene, DMF or mixtures thereof) and a suitable base (e.g. Na₂CO₃, NaHCO₃, KF, K₂CO₃ or TEA) at temperatures between room temperature and the boiling point of the solvent or solvent mixture, to yield intermediates 48 (step a).

Suzuki reactions of this type are broadly described in literature (e.g. A. Suzuki, Pure Appl. Chem. 1991, 63, 419-422; A. Suzuki, N. Miyaura, Chem. Rev. 1995, 95, 2457-2483; A. Suzuki, J. Organomet. Chem. 1999, 576, 147-168; V. Polshettiwar et al., Chem. Sus. Chem. 2010, 3, 502-522) and are well known to those skilled in the art. Alternatively, aryl- or heteroaryl-trifluoroborates 48c (FG=BF₃) can be used in the cross-coupling reaction applying a palladium catalyst such as, e.g. tetrakis(triphenylphosphine)-palladium(0), palladium(II) acetate or dichloro[1,1′-bis(diphenylphosphino)ferrocene]-palladium(II) dichloromethane adduct in the presence of a suitable base such as cesium carbonate or potassium phosphate in solvents such as toluene, THF, dioxane, water or mixtures thereof, at temperatures between room temperature and the boiling point of the solvent or solvent mixture (step a).

Alternatively, intermediates 47 can be reacted with aryl or heteroaryl stannanes 48d in which

FG is Sn(alkyl)₃ and alkyl is preferable n-butyl or methyl, using a suitable catalyst and solvent such as, e.g. tetrakis(triphenylphosphine)-palladium(0) in DMF at temperatures between room temperature and the boiling point of the solvent or solvent mixture to provide intermediates 49 (step a). Stille reactions of that type are well known in the art and described in literature, e.g. Org. React. 1997, 50, 1-652, ACS Catal. 2015, 5, 3040-3053.

Furthermore, intermediates 47 can be reacted with aryl or heteroarylzinc halides 48e in which FG is ZnHal and Hal preferably bromide or iodide, either commercially available or prepared by literature methods, using an appropriate catalyst and solvent system such as, e.g. [1,1′-bis(diphenylphosphino)ferrocene]dichloropalladium(II) and copper(I)iodide in DMA, or tetrakis(triphenylphosphine)palladium(0) in THF or DMF at temperatures between room temperature and the boiling point of the solvent to provide intermediates 49 (step a). Negishi reactions of that type are well known in the art and also described in literature, e.g. Org. Lett., 2005, 7, 4871, ACS Catal. 2016, 6 (3), 1540-1552. Acc. Chem. Res. 1982, 15 (11), pp 340-348. Alternatively, intermediates 49 may be prepared by converting intermediates 47 in which X is for example iodide into the corresponding zinc species by applying literature methods (e.g. reaction of 47 with Zn powder in the presence of chlorotrimethylsilane and 1,2-dibromoethane in a suitable solvent such as DMA) and coupling of the zinc species with aryl- or heteroarylbromides- or iodides under the conditions mentioned before.

Alternatively, intermediates 47 in which X is preferably bromide can be subjected to a cross-electrophile coupling with aryl- or heteroarylbromides 48f in which FG signifies bromide under irradiation with a 420 nm blue light lamp using an appropriate photo catalyst such as [Ir{dF(CF₃)ppy}2(dtbpy)]PF₆ ([4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate), a Nickel catalyst like NiCl₂ glyme (dichloro(dimethoxyethane)nickel), 4,4′-di-tert-butyl-2,2′-dipyridyl and tris(trimethylsilyl)silane, in the presence of a suitable base such as anhydrous sodium carbonate in a solvent like DME. Reactions of this type are described in literature, e.g. J. Am. Chem. Soc. 2016, 138, 8084. (step a).

Furthermore, intermediates 47 in which LG is preferably iodine can be subjected to Suzuki-Miyaura cross coupling reaction with arylboronic acids 50 (FG=B(OH)₂) using a suitable Nickel catalyst such as nickel(II) iodide in the presence of rac-(1R,2R)-2-aminocyclohexan-1-ol and a suitable base such as sodium bis(trimethylsilyl)amide in an appropriate solvent like iPrOH, dioxane, THF or DME, preferably iPrOH at temperatures between room temperature and the boiling point of the solvent, optionally applying microwave heating, to yield intermediates 51. Reactions of this type are described in literature, e.g. ChemistrySelect. 2017, 2, 8841 (step c).

Intermediates 51 can be reacted with compounds R⁴-FG 52 applying one of the cross-coupling methods described before to provide intermediates 49 (step d).

The bromo or iodo substituent in intermediates 51 can be converted into a boronic acid or boronic ester (e.g. pinacol ester) according to methods described in literature or as outlined under step a, to yield intermediates 53 (step e).

Intermediates 53 can be converted to intermediates 49 for example using Suzuki coupling with compounds R⁴-FG 52 in which FG is for example bromine or iodine applying the conditions described under step a (step f).

Removal of the protective group from intermediates 49 applying methods well known in the art and as described for example under Scheme 9, step b, furnishes intermediates 2c (step b).

In one aspect, the present invention provides a process of manufacturing the compounds of formula (I) described herein, comprising:
(a) reacting an amine of formula 2, wherein m, n, Q, L, A, R³ and R⁴ are as described herein,

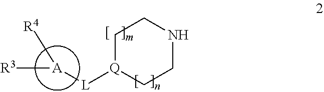

with a carboxylic acid 3a, wherein U, V, W, X, R¹ and R² are as described herein

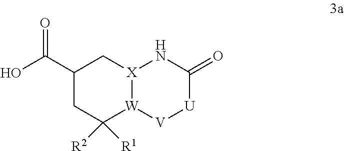

in the presence of a coupling reagent, such as CDI, DCC, HATU, HBTU, HOBT, TBTU, T3P or Mukaiyama reagent, and optionally in the presence of a base, such as TEA, DIPEA (Huenig's base) or DMAP; or
(b) reacting an amine of formula 2, wherein m, n, Q, L, A, R³ and R⁴ are as described herein,

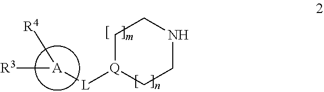

with a carboxylic acid chloride 3b, wherein U, V, W, X, IV and R² are as described herein

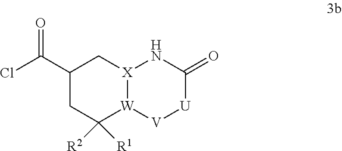

in the presence of a base, such as TEA, Huenig's base, pyridine, DMAP or lithium bis(trimethylsilyl)amide; or
(c) reacting a first amine of formula 1, wherein U, V, W, X, R¹ and R² are as described herein,

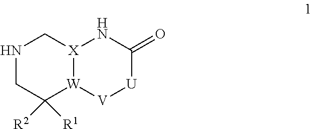

with a second amine 2, wherein A, L, m, n, Q, R³ and R⁴ are as described herein

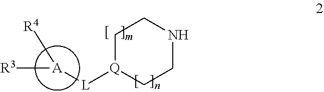

in the presence of a base, such as sodium bicarbonate, and a urea forming reagent, such as bis(trichloromethyl) carbonate, phosgene, trichloromethyl chloroformate, (4-nitrophenyl)carbonate or 1,1′-carbonyldiimidazole,
   to form said compound of formula (I).

In one aspect, the present invention provides a compound of formula (I) as described herein, when manufactured according to any one of the processes described herein.

### MAGL Inhibitory Activity

Compounds of the present invention are MAGL inhibitors. Thus, in one aspect, the present invention provides the use of compounds of formula (I) as described herein for inhibiting MAGL in a mammal.

In a further aspect, the present invention provides compounds of formula (I) as described herein for use in a method of inhibiting MAGL in a mammal.

In a further aspect, the present invention provides the use of compounds of formula (I) as described herein for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds were profiled for MAGL inhibitory activity by determining the enzymatic activity by following the hydrolysis of the natural substrate 2-arachidonoylglycerol resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 pM. 0.25 μL compound dilutions (100% DMSO) were added to 9 μL MAGL in assay buffer (50 mM TRIS (GIBCO, 15567-027), 1 mM EDTA (Fluka, 03690-100 ml), 0.01% (v/v) Tween. After shaking, the plate was incubated for 15 min at RT. To start the reaction, 10 μL 2-arachidonoylglycerol in assay buffer was added. The final concentrations in the assay was 50 pM MAGL and 8 μM 2-arachidonylglycerol. After shaking and 30 min incubation at RT, the reaction was quenched by the addition of 40 μL of acetonitrile containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC₅₀ MAGL |
|---|---|
| Example | [nM] |
| 1 | 4.4 |
| 2 | 117.6 |
| 3 | 11.1 |
| 4 | 1369 |
| 5 | 2.6 |
| 6 | 649.4 |
| 7 | 51.8 |
| 8 | 3.0 |
| 9 | 14.3 |
| 10 | 0.06 |
| 11 | 3.1 |
| 12 | 72.8 |
| 13 | 159 |
| 14 | 1430 |
| 15 | n/a |
| 16 | 791 |
| 17 | 48.3 |
| 18 | 1100 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

### Using the Compounds of the Invention

In one aspect, the present invention provides compounds of formula (I) as described herein for use as therapeutically active substance.

In a further aspect, the present invention provides the use of compounds of formula (I) as described herein for the treatment or prophylaxis of neuroinflammation, neurodegenerative diseases, pain, cancer, mental disorders and/or inflammatory bowel disease in a mammal.

In one embodiment, the present invention provides the use of compounds of formula (I) as described herein for the treatment or prophylaxis of neuroinflammation and/or neurodegenerative diseases in a mammal.

In one embodiment, the present invention provides the use of compounds of formula (I) as described herein for the treatment or prophylaxis of neurodegenerative diseases in a mammal.

In one embodiment, the present invention provides the use of compounds of formula (I) as described herein for the treatment or prophylaxis of cancer in a mammal.

In one embodiment, the present invention provides the use of compounds of formula (I) as described herein for the treatment or prophylaxis of inflammatory bowel disease in a mammal.

In one embodiment, the present invention provides the use of compounds of formula (I) as described herein for the treatment or prophylaxis of pain in a mammal.

In one aspect, the present invention provides the use of compounds of formula (I) as described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I) as described herein for the treatment or prophylaxis of multiple sclerosis, Alzheimer's disease and/or Parkinson's disease in a mammal.

In a particularly preferred embodiment, the present invention provides the use of compounds of formula (I) as described herein for the treatment or prophylaxis of multiple sclerosis in a mammal.

In one aspect, the present invention provides compounds of formula (I) as described herein for use in the treatment or prophylaxis of neuroinflammation, neurodegenerative diseases, pain, cancer, mental disorders and/or inflammatory bowel disease in a mammal.

In one embodiment, the present invention provides compounds of formula (I) as described herein for use in the treatment or prophylaxis of neuroinflammation and/or neurodegenerative diseases in a mammal.

In one embodiment, the present invention provides compounds of formula (I) as described herein for use in the treatment or prophylaxis of cancer in a mammal.

In one embodiment, the present invention provides compounds of formula (I) as described herein for use in the treatment or prophylaxis of neurodegenerative diseases in a mammal.

In one embodiment, the present invention provides compounds of formula (I) as described herein for use in the treatment or prophylaxis of inflammatory bowel disease in a mammal.

In one embodiment, the present invention provides compounds of formula (I) as described herein for use in the treatment or prophylaxis of pain in a mammal.

In one aspect, the present invention provides compounds of formula (I) as described herein for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides compounds of formula (I) as described herein for use in the treatment or prophylaxis of multiple sclerosis, Alzheimer's disease and/or Parkinson's disease in a mammal.

In a particularly preferred embodiment, the present invention provides compounds of formula (I) as described herein for use in the treatment or prophylaxis of multiple sclerosis in a mammal.

In one aspect, the present invention provides the use of compounds of formula (I) as described herein for the preparation of a medicament for the treatment or prophylaxis of neuroinflammation, neurodegenerative diseases, pain, cancer, mental disorders and/or inflammatory bowel disease in a mammal.

In one embodiment, the present invention provides the use of compounds of formula (I) as described herein for the preparation of a medicament for the treatment or prophylaxis of neuroinflammation and/or neurodegenerative diseases in a mammal.

In one embodiment, the present invention provides the use of compounds of formula (I) as described herein for the preparation of a medicament for the treatment or prophylaxis of neurodegenerative diseases in a mammal.

In one embodiment, the present invention provides the use of compounds of formula (I) as described herein for the preparation of a medicament for the treatment or prophylaxis of cancer in a mammal.

In one embodiment, the present invention provides the use of compounds of formula (I) as described herein for the preparation of a medicament for the treatment or prophylaxis of inflammatory bowel disease in a mammal.

In one embodiment, the present invention provides the use of compounds of formula (I) as described herein for the preparation of a medicament for the treatment or prophylaxis of pain in a mammal.

In a further aspect, the present invention provides the use of compounds of formula (I) as described herein for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I) as described herein for the preparation of a medicament for the treatment or prophylaxis of multiple sclerosis, Alzheimer's disease and/or Parkinson's disease in a mammal.

In a particularly preferred embodiment, the present invention provides the use of compounds of formula (I) as described herein for the preparation of a medicament for the treatment or prophylaxis of multiple sclerosis in a mammal.

In one aspect, the present invention provides a method for the treatment or prophylaxis of neuroinflammation, neurodegenerative diseases, pain, cancer, mental disorders and/or inflammatory bowel disease in a mammal, which method comprises administering an effective amount of a compound of formula (I) as described herein to the mammal.

In one embodiment, the present invention provides a method for the treatment or prophylaxis of neuroinflammation and/or neurodegenerative diseases in a mammal, which method comprises administering an effective amount of a compound of formula (I) as described herein to the mammal.

In one embodiment, the present invention provides a method for the treatment or prophylaxis of neurodegenerative diseases in a mammal, which method comprises administering an effective amount of a compound of formula (I) as described herein to the mammal.

In one embodiment, the present invention provides a method for the treatment or prophylaxis of cancer in a mammal, which method comprises administering an effective amount of a compound of formula (I) as described herein to the mammal.

In one embodiment, the present invention provides a method for the treatment or prophylaxis of inflammatory bowel disease in a mammal, which method comprises administering an effective amount of a compound of formula (I) as described herein to the mammal.

In one embodiment, the present invention provides a method for the treatment or prophylaxis of pain in a mammal, which method comprises administering an effective amount of a compound of formula (I) as described herein to the mammal.

In a further aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal, which method comprises administering an effective amount of a compound of formula (I) as described herein to the mammal.

In a preferred embodiment, the present invention provides a method for the treatment or prophylaxis of multiple sclerosis, Alzheimer's disease and/or Parkinson's disease in a mammal, which method comprises administering an effective amount of a compound of formula (I) as described herein to the mammal.

In a particularly preferred embodiment, the present invention provides a method for the treatment or prophylaxis of multiple sclerosis in a mammal, which method comprises administering an effective amount of a compound of formula (I) as described herein to the mammal.

### Pharmaceutical Compositions and Administration

In one aspect, the present invention provides a pharmaceutical composition comprising a compound of formula (I) as described herein and a therapeutically inert carrier.

In one embodiment, the present invention provides the pharmaceutical compositions disclosed in Examples 19 and 20.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### rel-(4aR,8S,8aS)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one

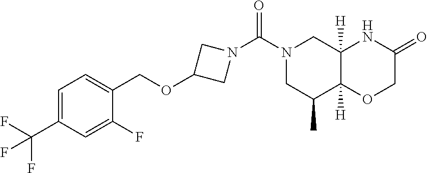 [SMILES: C[C@H]1CN(C(=O)N2CC(OCc3ccc(C(F)(F)F)cc3F)C2)C[C@H]2NC(=O)CO[C@@H]12]

To an ice-cold solution of bis(trichloromethyl) carbonate (34.2 mg, 115 μmol) in DCM (2 mL) ere added sodium bicarbonate (55.3 mg, 658 μmol) and rel-(4aR,8S,8aS)-8-methylhexahydro-2H-pyrido[4,3-b][1,4]oxazin-3(4H)-one (35 mg, 165 μmol) and the mixture was stirred at RT overnight. To the suspension was added 3-((2-fluoro-4-(trifluoromethyl)benzyl)oxy)azetidine 4-methylbenzenesulfonate (69.3 mg, 165 μmol) and DIPEA (85 mg, 115 μL, 658 μmol). The suspension was stirred at RT for 1.5 h. The reaction mixture was poured on water and DCM and the layers were separated. The aqueous layer was extracted three times with DCM. The organic layers were washed twice with water, dried over MgSO₄, filtered, and evaporated. The compound was purified by prep HPLC to provide the desired compound as a white solid. M/Z (ESI) 446.3 [M+H]⁺.

### Intermediate

### 3-((2-Fluoro-4-(trifluoromethyl)benzyl)oxy)azetidine 4-methylbenzenesulfonate

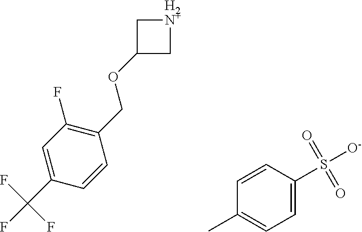

To an ice-cold solution of tert-butyl 3-((2-fluoro-4-(trifluoromethyl)benzyl)oxy)azetidine-1-carboxylate (7.8 g, 22.3 mmol) in EtOAc (130 mL) was added 4-methylbenzenesulfonic acid monohydrate (4.61 g, 26.8 mmol) and the mixture was heated at reflux for 3 h. The rapidly formed suspension was allowed to cool down to RT overnight. The suspension was filtered, the filter cake was washed with EtOAc (20 mL) to provide the desired product as a colorless solid (7.3 g; 81.2%). MS (ESI): m/z=250.2 [M+H]⁺.

### Step a) tert-Butyl 3-((2-fluoro-4-(trifluoromethyl)benzyl)oxy)azetidine-1-carboxylate

To an ice-cold solution of tert-butyl 3-hydroxyazetidine-1-carboxylate (2.02 g, 11.7 mmol, CAS RN 141699-55-0) in DMF (25 mL) was added NaH (55% dispersion in mineral oil; 560 mg, 12.8 mmol) in portions and the mixture was stirred at 0° C. for 30 min. A solution of 1-(bromomethyl)-2-fluoro-4-(trifluoromethyl)benzene (3 g, 11.7 mmol, CAS RN 239087-07-1) in DMF (5 mL) was added dropwise to the mixture. Stirring of the slurry was continued at RT for 3 h. Then the reaction mixture was poured on saturated aq. NH₄Cl solution (70 mL) and EtOAc (70 mL) and the layers were separated. The aqueous layer was extracted once with EtOAc (50 mL). The organic layers were washed twice with water, dried over MgSO₄, filtered, treated with silica gel and evaporated. The compound was purified by silica gel chromatography on a 40 g column using an MPLC system eluting with a gradient of n-heptane:EtOAc (100:0 to 60:40) to provide the desired compound as a light yellow oil (3.66 g; 89.8%). MS (ESI): m/z=294.1 [M−56+H]⁺.

### Example 2

### rel-(4aS,8R,8aR)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one

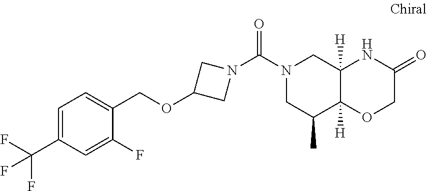 [SMILES: C[C@H]1CN(C(=O)N2CC(OCc3ccc(C(F)(F)F)cc3F)C2)C[C@H]2NC(=O)CO[C@@H]12]

To an ice-cold solution of bis(trichloromethyl) carbonate (34.2 mg, 115 μmol) in DCM (2 mL) were added sodium bicarbonate (55.3 mg, 658 μmol) and rel-(4aS,8R,8aR)-8-methylhexahydro-2H-pyrido[4,3-b][1,4]oxazin-3(4H)-one (35 mg, 165 μmol) and the mixture was stirred at RT overnight. To the suspension was added 3-((2-fluoro-4-(trifluoromethyl)benzyl)oxy)azetidine 4-methylbenzenesulfonate (69.3 mg, 165 μmol, example 1, intermediate) and DIPEA (85 mg, 115 μL, 658 μmol). The suspension was stirred at RT for 1.5 h. The reaction mixture was poured on water and DCM and the layers were separated. The aqueous layer was extracted three times with DCM. The organic layers were washed twice with water, dried over MgSO₄, filtered, and evaporated. The compound was purified by prep-HPLC to yield the desired compound as a white solid. MS (ESI): m/z=446.3 [M+H]⁺.

### Step a) N-(5-bromo-4-hydroxy-3-pyridyl)-2-chloro-acetamide

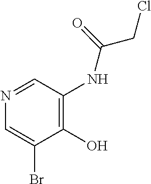 [SMILES: O=C(CCl)Nc1cncc(Br)c1O]

To an ice-cold suspension of 3-amino-5-bromopyridin-4-ol (2 g, 10.6 mmol) and sodium acetate trihydrate (2.88 g, 21.2 mmol) in acetone (80 mL) and water (6 mL) was added dropwise a solution of 2-chloroacetyl chloride (1.25 g, 885 μL, 11.1 mmol) in acetone (5 mL). The mixture was stirred at RT for 18 h, then acetone and water was removed under high vacuum. The crude material was purified by silica gel chromatography using a gradient of DCM:MeOH (100:0 to 85:15) to yield the desired product as light brown solid (82%). MS (ESI)=267.1[M+H]⁺.

### Step b) 8-Bromo-4H-pyrido[4,3-b][1,4]oxazin-3-one

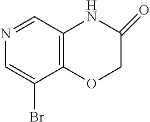 [SMILES: O=C1COc2c(Br)cncc2N1]

To a solution of N-(5-bromo-4-hydroxypyridin-3-yl)-2-chloroacetamide (2.3 g, 8.66 mmol) in DMF (45 mL) was added K₂CO₃ (2.39 g, 17.3 mmol), the suspension was heated to 100° C. and stirred for 1 h. The mixture was filtered to remove the K₂CO₃ and the filtrate was evaporated. To the remaining solid EtOAc (50 mL) and water (20 mL) were added. The solution was shaked a couple of times and the precipitated product was filtered off. The filtrate was extracted until the aqueous phase didn't show any trace of product anymore. The organic phases were combined, dried with MgSO₄ and concentrated under vacuum to provide the desired product as an off-white solid (72%). MS (ESI): m/z=231.0 [M+H]⁺.

### Step c) 8-Methyl-4H-pyrido[4,3-b][1,4]oxazin-3-one

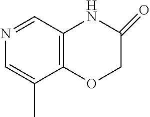

A 25 mL tube was charged with 8-bromo-2H-pyrido[4,3-b][1,4]oxazin-3(4H)-one (350 mg, 1.53 mmol), K₂CO₃ (317 mg, 2.29 mmol), tetrakis(triphenylphosphine)palladium(0) (88.3 mg, 76.4 μmol) and flushed with argon. Degassed dioxane (8.2 mL) and trimethylboroxine (269 mg, 299 μL, 2.14 mmol) were added, the mixture kept for 2 min in an ultrasonic bath, then water was added (2.7 mL) and the mixture kept for another 2 min in an ultrasonic bath. The yellow suspension was stirred at 135° C. for 24 h upon which a clear yellow solution formed. After cooling down to 20° C. (without stirring), the solid material was filtered off and washed with 5 mL EtOAc to provide 100 mg of white needles. The mother liquor was removed under vacuum, the residue stirred in a mixture of 30 mL DCM:MeOH (9:1) for 20 min, filtered and the organic solvent removed under vacuum. The residue was crystallized from hot dioxane to give 30 mg product. The product batches were combined to yield 130 mg of the desired product as an off-white solid. MS (ESI): m/z=165.1 [M+H]⁺.

### Step d) (4aR, 8S or 8R,8aS)-8-Methyl-4a,5,6,7,8,8a-hexahydro-4H-pyrido[4,3-b][1,4]oxazin-3-one and (4aS, 8R or 8S,8aR)-8-methyl-4a,5,6,7,8,8a-hexahydro-4H-pyrido[4,3-b][1,4]oxazin-3-one

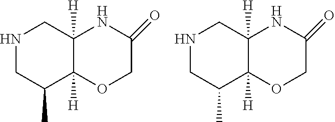

In a high pressure reactor, 350 mg of 8-methyl-2H-pyrido [4,3-b] [1,4] oxazine-3 (4H)-one (2.13 mmol) were suspended in 7 mL of MeOH. 114 μL sulfuric acid (2.13 mmol) and 350 mg (68 mmol) of Rh/C (5% wet; water 59.4%) Noblyst P3053 #2514 were added. The apparatus was closed and the reaction mixtures was hydrogenated under 50 bar of hydrogen pressure at 50° C. for 18 h. The suspension was filtered and the filtrate was evaporated to provide 230 mg of a yellow residue. The residue was further purified by chiral separation (Chiralcel OD, Flow: 40 mL/min; 207 nm, (70% n-heptane/30% EtOH+0.05% NH₄OAc) to yield the two enantiomers of the desired compound.

Enantiomer A (first eluting): 70 mg yellow solid, MS (ESI): m/z=170.1 [M+H]⁺;

Enantiomer B (second eluting): 68 mg yellow solid, MS (ESI): m/z=170.1 [M+H]⁺.

### Example 3

### rel-(4aS,8aS)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one

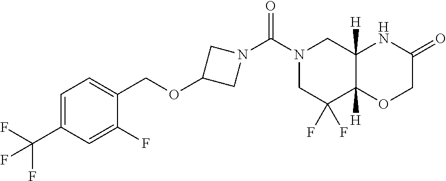 [SMILES: O=C1CO[C@H]2[C@H](CN(C(=O)N3CC(OCc4ccc(C(F)(F)F)cc4F)C3)CC2(F)F)N1]

To a solution of tert-butyl rel-(4a5,8a5)-8,8-difluoro-3-oxohexahydro-2H-pyrido[4,3-b][1,4]oxazine-6(5H)-carboxylate (enantiomer A, 38 mg, 130 μmol) in dry DCM (2 mL) under argon was added TFA (119 mg, 80.1 μL, 1.04 mmol) and the reaction stirred at RT for 4 h. The solvent was removed under vacuum, the residue dissolved in 2 mL ACN and TEA (92.1 mg, 127 μL, 910 μmol) was added. Then, 1,1′-carbonyl-di(1,2,4-triazole) (21.3 mg, 130 μmol) was added and the reaction mixture stirred for 60 min. Then 3-((2-fluoro-4-(trifluoromethyl)benzyl)oxy)azetidine 4-methylbenzenesulfonate (65.7 mg, 156 μmol, example 1, intermediate) were added and the reaction stirred at RT for 4 h. The reaction mixture was quenched with 2 mL water, extracted twice with EtOAc (10 mL each), 4 mL 5% aqueous NaHCO₃ solution, 4 mL 0.5N HCl and brine. The organic layer was separated, dried over Na₂SO₄ and evaporated. The residue was purified by prep-HPLC (Gemini NX column, 12 nm, 5 μm, 100×30 mm, ACN/water+0.1% HCOOH) and the pooled fractions containing to the product lyophilized to yield the desired compound (43%). MS (ESI): m/z=468.2 [M−56+H]⁺.

### Example 4

### rel-(4aR,8aR)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one

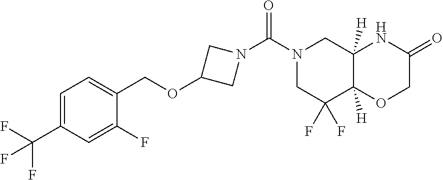 [SMILES: O=C1CO[C@@H]2[C@@H](CN(C(=O)N3CC(OCc4ccc(C(F)(F)F)cc4F)C3)CC2(F)F)N1]

In a 20 mL glastube under argon, tert-butyl rel-(4aR,8aR)-8,8-difluoro-3-oxohexahydro-2H-pyrido[4,3-b][1,4]oxazine-6(5H)-carboxylate (enantiomer B, 38 mg, 130 μmol) was dissolved in dry DCM (2 mL). TFA (119 mg, 80.1 μL, 1.04 mmol) was added and the solution stirred at RT for 4 h before the volatiles were removed. The residue was dissolved in 2 mL ACN and TEA (92.1 mg, 127 μL, 910 μmol) was added. Then, 1,1′-carbonyl-di(1,2,4-triazole) (21.3 mg, 130 μmol) were added and the reaction mixture stirred for 60 min. Then, 3-((2-fluoro-4-(trifluoromethyl)benzyl)oxy)azetidine 4-methylbenzenesulfonate (65.7 mg, 156 μmol, example 1, intermediate) were added and stirring continued for 4 h. The reaction mixture was quenched with 2 mL water and extracted twice with EtOAc (10 mL each), 4 mL 5% aqueous NaHCO₃ solution, 4 mL 0.5N HCl and brine. The organic layer was separated, dried over Na₂SO₄ and evaporated. The crude product was purified by prep-HPLC (Gemini NX column, 12 nm, 5 μm, 100×30 mm, ACN/Water+0.1% HCOOH), the product-containing fractions pooled and lyophilized to provide the title compound (43%). MS (ESI): m/z=468.2 [M−56+H]⁺.

### Step a) 1-tert-Butyl 3-ethyl rac-(3R,4R)-5,5-difluoro-4-hydroxy-piperidine-1,3-dicarboxylate

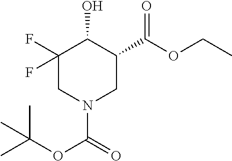 [SMILES: CCOC(=O)[C@@H]1CN(C(=O)OC(C)(C)C)CC(F)(F)[C@@H]1O]

In a sulfonating flask was successively added 1-(tert-butyl) 3-ethyl-5,5-difluoro-4-oxopiperidine-1,3-dicarboxylate (5 g, 15.8 mmol), dissolved in isopropanol (19.6 g, 25 mL, 325.9 mmol), potassium phosphate buffer 1 M, pH 7.0 (50 mL, 50 mmol), water (310 g, 310 mL, 17.21 mol), D (+)-glucose monohydrate from a 1 M stock solution in dH2O (100 mL, 100 mmol), MgCl₂×6 H₂O from a 100 mM stock solution in dH2O (10 mL, 1 mmol) and NADP+ disodium salt (50 mg, 63.5 μmol). The mixture was stirred at RT for 5 min. Glucose dehydrogenase (GDH-105, Codexis) (50 mg) and Ketoreductase 130 (KRED 130, Codexia) (500 mg) was added.

The pH was kept constant (pH at start 7.05) over the reaction time using a pH Stat (902 Titrando, Metrohm) adding NaOH (1 M, 15.78 mL, 15.78 mmol). The reaction was stopped after 18 h by addition of 250 mL EtOAc, vigorously stirred for 5 min. and then the 2-phase mixture was rinsed in a Schott bottle. Dicalite (30 g) was added to the reaction mixture, stirred for 15 min and then filtered over a dicalite cake (30 g). The 2-phase mixture was separated in a separating funnel, and the water phase extracted 3 times with EtOAc (250 mL each). The EtOAc layer was dried over MgSO₄, filtered, the filtrate completely concentrated under vacuo at 40° C. and the residue dried at 40° C./<5 mbar for 1 h. Light yellow viscous oil (4.37 g, 89%). MS (ESI): m/z=254.2 [M−56+H]⁺.

### Step b) rac-(3R,4R)-1-tert-butoxycarbonyl-5,5-difluoro-4-hydroxy-piperidine-3-carboxylic Acid

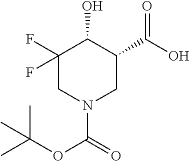 [SMILES: CC(C)(C)OC(=O)N1C[C@@H](C(=O)O)[C@@H](O)C(F)(F)C1]

1-(tert-Butyl) 3-ethyl 5,5-difluoro-4-hydroxypiperidine-1,3-dicarboxylate (500 mg, 1.62 mmol) was dissolved in MTBE (1.04 g, 1.41 mL, 11.8 mmol). To the clear colorless solution NaOH (3.23 mL, 6.47 mmol) was added over 10 min and the biphasic mixture was vigorously stirred at RT for 90 min. The reaction mixture was transferred into a separation funnel and the aq. layer was separated, acidified with 2 mL 25% HCl (pH=1, ice bath cooling) and transferred to a separation funnel. Then the aq. layer was extracted twice with TBME (10 mL each) and the organic layers were concentrated in vacuo to yield the desired compound as a white foam (94%). MS (ESI): m/z=280.2 [M−H]⁻.

### Step c) tert-Butyl rac-(3aS,7aS)-7,7-difluoro-2-oxo-3a,4,6,7a-tetrahydro-3H-oxazolo[4,5-c]pyridine-5-carboxylate

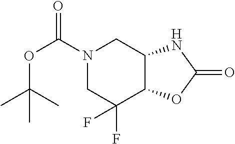 [SMILES: CC(C)(C)OC(=O)N1C[C@@H]2NC(=O)O[C@@H]2C(F)(F)C1]

1-(tert-Butoxycarbonyl)-5,5-difluoro-4-hydroxypiperidine-3-carboxylic acid (1300 mg, 4.62 mmol) was suspended in dry toluene (3.83 g, 4.43 mL, 41.6 mmol) and TEA (1.4 g, 1.93 mL, 13.9 mmol) was added. The resulting clear colorless solution was heated to 82° C. under stirring. Then diphenylphosphoryl azide 97% (1.44 g, 1.13 mL, 5.08 mmol) was added dropwise over 10 min. The reaction mixture was stirred at 80° C. for 1.5 h. The light yellow reaction mixture was cooled down to RT and 2.5 mL 1M NaOH were added. After stirring at RT for 10 min, the reaction mixture was extracted with EtOAc and water, the organic layer dried over MgSO₄ and the solvent removed under vacuum. The residue was purified by silica gel chromatography using a gradient of EtOAc:n-heptane (0:100 to 100:0) to yield the desired product as a light yellow solid (22%). MS (ESI): m/z=277.2 [M−H]⁻.

### Step d) tert-Butyl rac-(4R,5R)-5-amino-3,3-difluoro-4-hydroxy-piperidine-1-carboxylate

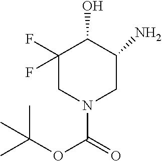 [SMILES: CC(C)(C)OC(=O)N1C[C@@H](N)[C@@H](O)C(F)(F)C1]

tert-Butyl rac-7,7-difluoro-2-oxohexahydrooxazolo[4,5-c]pyridine-5(4H)-carboxylate (275 mg, 988 μmol) was suspended in cyclopentyl methyl ether (1.48 g, 1.73 mL, 14.8 mmol) and NaOH (1.88 mL, 3.76 mmol) was added at 22° C. The two layer suspension was heated to 70° C. and stirred for 6 h. Then the reaction mixture was cooled to RT and transferred into a separation funnel (5 mL CPME were used for transfer). The layers were separated, the aqueous layer was extracted twice with CPME (6 mL each) followed by brine. The organic layers were evaporated to furnish the compound as a white solid (57%). MS (ESI): m/z=253.2 [M−H]⁻.

### Step e) tert-Butyl rac-(4R,5R)-5-[(2-chloroacetyl)amino]-3,3-difluoro-4-hydroxy-piperidine-1-carboxylate

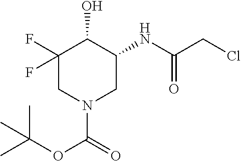 [SMILES: CC(C)(C)OC(=O)N1C[C@@H](NC(=O)CCl)[C@@H](O)C(F)(F)C1]

tert-Butyl 5-amino-3,3-difluoro-4-hydroxypiperidine-1-carboxylate (142 mg, 563 μmol) was dissolved in isopropyl acetate (1.74 g, 2 mL, 17 mmol) at 50° C., then cooled to RT and a solution of Na₂CO₃ (89.5 mg, 844 μmol) in water (1 g, 1 mL, 55.5 mmol) was added. The resulting clear biphasic solution was cooled to 0° C. and chloroacetyl chloride (80.7 mg, 56.9 μL, 715 μmol) was slowly added dropwise at 0-4° C. The reaction mixture was stirred at 0° C. for 15 min, warmed to RT, quenched with 5 mL water and transferred to a separation funnel. The layers were separated and the aq. layer was reextracted twice with EtOAc (10 mL each). The combined organic layers were dried over MgSO₄ and evaporated to yield the title compound as a light yellow oil (97%). MS (ESI): m/z=273.1 [M−56+H]⁺.

### Step f) tert-Butyl rel-(4aS,8aS)-8,8-difluoro-3-oxo-4a,5,7,8a-tetrahydro-4H-pyrido[4,3-b][1,4]oxazine-6-carboxylate and tert-Butyl rel-(4aR,8aR)-8,8-difluoro-3-oxo-4a,5,7,8a-tetrahydro-4H-pyrido[4,3-b][1,4]oxazine-6-carboxylate

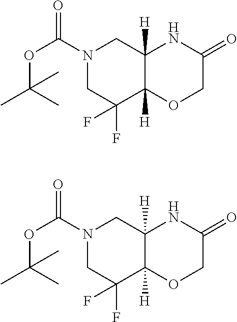 [SMILES: CC(C)(C)OC(=O)N1C[C@@H]2NC(=O)CO[C@@H]2C(F)(F)C1 ; CC(C)(C)OC(=O)N1C[C@H]2NC(=O)CO[C@H]2C(F)(F)C1]

Potassium tert-butoxide (246 mg, 2.19 mmol) was dissolved portion-wise in 2.5 mL 2-propanole at 0-5° C. The solution was warmed to 30° C. and tert-butyl rac-5-(2-chloroacetamido)-3,3-difluoro-4-hydroxypiperidine-1-carboxylate (180 mg, 548 μmol) in 1.6 mL 2-propanol was added in one portion. The reaction mixture was stirred at 30-35° C. for 30 min. The reaction mixture was allowed to cool to 18-20° C., quenched with 1 mL water and neutralized with 0.9 mL 2M HCl (pH=6), then concentrated in vacuo. The residue was extracted three times with EtOAc (10 mL each). The organic layers were dried with MgSO₄ and evaporated. The residue was purified and the enantiomers separated by SFC (OD-H column, 12 nm, 5 μm, 250×20 mm, 10% EtOH) to provide the title compounds.

Enantiomer A (first eluting): 38 mg (24%), colorless viscous oil, MS (ESI) m/z=237.2 [M−56+H]⁺.

Enantiomer B (second eluting): 37 mg (23%), white foam, MS (ESI) m/z=237.2 [M−56+H]⁺.

### Example 5

### rel-(4aS,8aS)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one

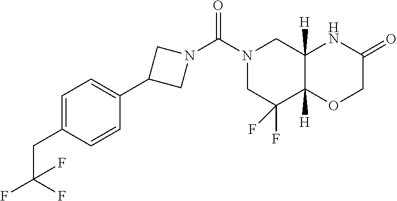 [SMILES: O=C1CO[C@H]2[C@H](CN(C(=O)N3CC(c4ccc(CC(F)(F)F)cc4)C3)CC2(F)F)N1]

In a glastube under argon, tert-butyl rel-(4aS,8aS)-8,8-difluoro-3-oxohexahydro-2H-pyrido[4,3-b][1,4]oxazine-6(5H)-carboxylate (enantiomer A, 0.025 g, 85.5 μmol) was dissolved in DCM (1.5 mL) and TFA (78 mg, 52.7 μL, 684 μmol) was added. The reaction mixture was stirred at RT for 1 h and the solvent was removed. The residue was dissolved in ACN (1.5 mL), TEA (60.6 mg, 83.5 μL, 599 μmol) was added followed by 1,1′-carbonyl-di(1,2,4-triazole) (16.8 mg, 103 μmol). The reaction mixture was stirred at RT, 3-(4-(2,2,2-trifluoroethyl)phenyl)azetidine 4-methylbenzenesulfonate (39.8 mg, 103 μmol) was added and stirring was continued at RT for 3 h. The reaction mixture was quenched with water and extracted twice with EtOAc. The organic layers were combined, washed with 5% aqueous NaHCO₃ solution followed by HCl 0.5M, dried over Na₂SO₄ and concentrated. The crude product was purified by flash chromatography (silica gel, 10 g, gradient MeOH:DCM 0:100 to 10:90) to yield the desired product as a white foam (73%). MS (ESI): m/z=434.3 [M+H]⁺.

### Intermediate

### 3-(4-(2,2,2-Trifluoroethyl)phenyl)azetidine 4-methylbenzenesulfonate

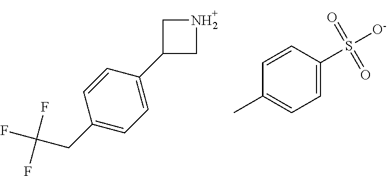

To a solution of tert-butyl 3-(4-(2,2,2-trifluoroethyl)phenyl)azetidine-1-carboxylate (975 mg, 3.09 mmol), in EtOAc (12 mL), 4-methylbenzenesulfonic acid (639 mg, 3.71 mmol) was added. The reaction mixture was heated to reflux and stirring continued for 2 h. After cooling down to RT the formed suspension was concentrated in vacuo affording the title compound as a colourless solid (540.3 mg; 45.1%). MS (ESI): m/z=216.1 [M+H]⁺.

### Step a) tert-Butyl 3-(4-(2,2,2-trifluoroethyl)phenyl)azetidine-1-carboxylate

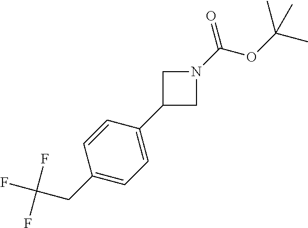 [SMILES: CC(C)(C)OC(=O)N1CC(c2ccc(CC(F)(F)F)cc2)C1]

To a 20 mL vial equipped with a stirring bar was added (Ir[dF(CF3)ppy]2(dtbpy))PF6 (23.8 mg, 21.2 μmol), 1-bromo-4-(2,2,2-trifluoroethyl)benzene (506 mg, 2.12 mmol), tert-butyl 3-bromoazetidine-1-carboxylate (500 mg, 2.12 mmol), 1,1,1,3,3,3-hexamethyl-2-(trimethylsilyl)trisilane (527 mg, 653 μL, 2.12 mmol) and anhydrous sodium carbonate (449 mg, 4.24 mmol). The vial was sealed and placed under argon before DME (9 mL) was added. To a separate vial was added nickel(II) chloride ethylene glycol dimethyl ether complex (4.65 mg, 21.2 μmol) and 4,4′-di-tert-butyl-2,2′-bipyridine (5.68 mg, 21.2 μmol). The precatalyst vial was sealed, purged with argon then DME (4 mL) was added. The precatalyst vial was sonicated for 5 min, after which, 2 mL of it was syringed into the 20 mL vial. The suspension was degassed with argon and the reaction was stirred and irradiated with a 420 nm lamp for 1 h. Then the reaction mixture was filtered and the filtrate was treated with silica gel and evaporated. The compound was purified first by silica gel chromatography on a 12 g column using an MPLC (ISCO) system eluting with a gradient of n-heptane:EtOAc (100:0 to 70:30) followed by silica gel chromatography on a 40 g column using an MPLC (ISCO) system eluting with an isocratic mixture of n-heptane:EtOAc (100:0 to 70:30) to yield the desired compound as a colorless liquid (0.297 g; 42.3%). MS (ESI): m/z=260.1 [M−56+H]⁺.

### Example 6

### rel-(4aR,8aR)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one

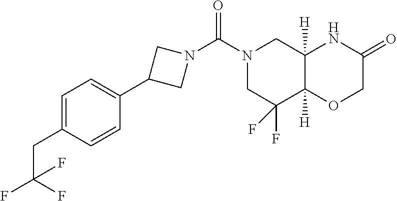 [SMILES: O=C1CO[C@@H]2[C@@H](CN(C(=O)N3CC(c4ccc(CC(F)(F)F)cc4)C3)CC2(F)F)N1]

In a glastube under argon, tert-butyl rel-(4aR,8aR)-8,8-difluoro-3-oxohexahydro-2H-pyrido[4,3-b][1,4]oxazine-6(5H)-carboxylate (enantiomer B, 0.032 g, 109 μmol) was dissolved in DCM (2 mL) and TFA (99.9 mg, 67.5 μL, 876 μmol) was added. the reaction mixture was stirred at RT for 2 h. The solvent was removed and the residue dissolved in ACN (2 mL). TEA (77.6 mg, 107 μL, 766 μmol) was added, followed by 1,1′-carbonyl-di(1,2,4-triazole) (21.6 mg, 131 μmol). The reaction mixture was stirred at RT for 1 h. Then 3-(4-(2,2,2-trifluoroethyl)phenyl)azetidine 4-methylbenzenesulfonate (50.9 mg, 131 μmol, example 5, intermediate) was added and stirring continued at RT for 3 h. The reaction mixture was quenched with water and extracted twice with EtOAc. The combined organic layers were washed with aqueous 5% NaHCO₃ solution, followed with HCl 0.5M, dried over Na₂SO₄, filtered and concentrated. The crude product was purified by prep-HPLC (Gemini NX column) using a gradient of ACN:water (containing 0.1% TEA) (15:85 to 100:0) to provide the desired compound as a white powder (16.6 mg; 35%). MS (ESI): m/z=424.3 [M+H]⁺.

### Example 7

### 6-[4-[[4-(Trifluoromethyl)phenyl]methyl]piperidine-1-carbonyl]-4,5,7,8-tetrahydropyrido[4,3-b][1,4]oxazin-3-one

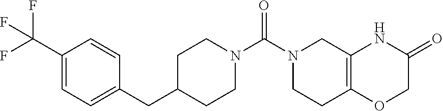 [SMILES: O=C1COC2=C(CN(C(=O)N3CCC(Cc4ccc(C(F)(F)F)cc4)CC3)CC2)N1]

To an ice-cold solution of bis(trichloromethyl) carbonate (84.7 mg, 286 μmol) in DCM (1.5 mL) were added sodium bicarbonate (137 mg, 1.63 mmol) and 4-[[4-(trifluoromethyl)phenyl]methyl]piperidine; hydrochloride (114 mg, 408 μmol. CAS RN 192990-03-7) and the mixture was stirred overnight at RT. To the suspension was added a solution of 5,6,7,8-tetrahydro-2H-pyrido[4,3-b][1,4]oxazin-3(4H)-one (74 mg, 408 μmol) in DCM (1.5 mL) and DIPEA (211 mg, 285 μL, 1.63 mmol). The suspension was stirred at RT for 1.75 h. The reaction mixture was poured on water and DCM and the layers were separated. The aqueous layer was extracted three times with DCM. The organic layers were washed twice with water, dried over MgSO₄, filtered, treated with silica gel and evaporated. The compound was purified by silica gel chromatography on a 4 g column using an MPLC system eluting with a gradient of n-heptane:EtOAc (100:0 to 0:100) to yield the crude product. The product was purified on a preparative HPLC (Gemini NX column) using a gradient of ACN:water (containing 0.1% TEA) (15:85 to 100:0) to provide the desired compound as a colorless solid (0.041 g; 23.7%). MS (ESI): m/z=424.3 [M+H]⁺.

### Step a) 6-Benzyl-4H-pyrido[4,3-b][1,4]oxazin-6-ium-3-one; Bromide

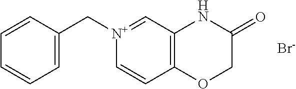 [SMILES: O=C1COc2cc[n+](Cc3ccccc3)cc2N1 ; [Br-]]

A suspension of 2H-pyrido[4,3-b][1,4]oxazin-3(4H)-one (4.0 g, 26.6 mmol) in DCM (42 mL) was treated with (bromomethyl)benzene (5.47 g, 3.8 mL, 32 mmol) and MeOH (10.4 mL) and the mixture was stirred at RT for 60 h. A suspension formed, which was cooled down to 4° C. and then filtered. The filtrate was washed with cold DCM/n-hexane to furnish the desired compound as a colorless solid (7.63 g; 89%). MS (ESI): m/z=241.1 [M+H]⁺.

### Step b) 6-Benzyl-4,5,7,8-tetrahydropyrido[4,3-b][1,4]oxazin-3-one

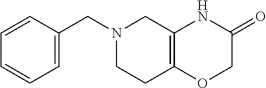 [SMILES: O=C1COC2=C(CN(Cc3ccccc3)CC2)N1]

To a suspension of 6-benzyl-3-oxo-3,4-dihydro-2H-pyrido[4,3-b][1,4]oxazin-6-ium bromide (7.6 g, 23.7 mmol) in EtOH (41 mL) was added in portions NaBH₄ (1.25 g, 33.1 mmol) (exothermic, 22° C. to 45° C., yellow suspension formed). The mixture was allowed to cool down to 22° C. over 2 h. The reaction mixture was then evaporated, partioned between water and EtOAc and the layers were separated. The aqueous layer was extracted once with EtOAc. The organic layers were washed twice with water, dried over MgSO₄, filtered, treated with silica gel and evaporated. The compound was purified by silica gel chromatography using an MPLC system eluting with a gradient of n-heptane:EtOAc (50:50 to 0:100 in 25 min) to yield the desired compound as an off-white solid (3.6 g; 62.3%). MS (ESI): m/z=245.2 [M+H]⁺.

### Step c) 5,6,7,8-Tetrahydro-4H-pyrido[4,3-b][1,4]oxazin-3-one

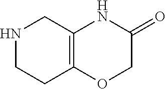 [SMILES: O=C1COC2=C(CNCC2)N1]

To a solution of 6-benzyl-5,6,7,8-tetrahydro-2H-pyrido[4,3-b][1,4]oxazin-3(4H)-one (100 mg, 409 μmol) in MeOH (1 mL) under argon was added acetyl chloride (32.1 mg, 29.1 μL, 409 μmol) followed by addition of Pd/C 10% (10 mg, 409 μmol). The suspension was stirred under a 1.5 bar hydrogen atmosphere at RT for 2.5 h. The mixture was filtered and the filter cake was washed with MeOH. The filtrate was evaporated to yield the desired compound as an off-white solid (0.074 g; 99.7%). MS (ESI): m/z=155.1 [M+H]⁺.

### Example 8

### 7-(4-Benzhydrylpiperidine-1-carbonyl)-1,5,6,8-tetrahydro-1,7-naphthyridin-2-one

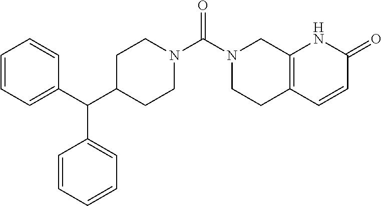 [SMILES: O=C(N1CCC(C(c2ccccc2)c2ccccc2)CC1)N1CCc2ccc(=O)[nH]c2C1]

To a mixture of 5,6,7,8-tetrahydro-1H-1,7-naphthyridin-2-one 2,2,2-trifluoroacetic acid salt (100.0 mg, 0.380 mmol) and DIEA (97.8 mg, 0.760 mmol) in DCM (3 mL) was added 4-benzhydrylpiperidine-1-carbonyl chloride (120.0 mg, 0.380 mmol, example 10, step h). The mixture was stirred at 20° C. for 12 h. The mixture was concentrated and the residue was purified by prep-HPLC (0.5% v/v ammonia in water and MeCN) to give the title compound as an off-white solid (20 mg, 0.050 mmol, 12.1%). MS (ESI): m/z=428.3 [M+H]⁺.

### Step a) Ethyl (E)-3-(4,4,5,5-tetramethyl-1,3,2-dioxaborolan-2-yl)prop-2-enoate

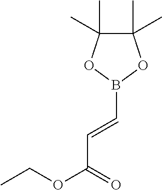 [SMILES: CCOC(=O)/C=C/B1OC(C)(C)C(C)(C)O1]

A solution of copper(l) chloride (158.71 mg, 1.6 mmol) sodium t-butanolate (462.2 mg, 4.81 mmol) and (5-diphenylphosphanyl-9,9-dimethyl-xanthen-4-yl)-diphenyl-phosphane (927.62 mg, 1.6 mmol) in THF (65 mL) was purged with N₂, and stirred at 25° C. for 30 min. Then, bis(pinacolato)diboron (13.6 g, 53.4 mmol) in THF (33 mL) was added, and the reaction stirred for another 10 min. Ethyl propiolate (5.4 mL, 53.4 mmol, CAS RN 623-47-2) was added followed by MeOH (4 mL). The reaction mixture was stirred at 25° C. for 11 h. The reaction was quenched with water (50 mL) and after removal of MeOH extracted three times with EtOAc (100 mL each). The combined organic layers were washed twice with water (40 mL each) and brine (40 mL), dried over Na₂SO₄ and concentrated under reduced pressure. The residue was purified by column chromatography (PE:EtOAc=1:0 to 5:1) to yield the desired compound as a light yellow oil (10.3 g, 45.56 mmol, 85.3%). Proton NMR: ¹H NMR (300 MHz, CHLOROFORM-d) δ=6.69-6.60 (m, 1H), 6.55-6.44 (m, 1H), 4.09 (q, J=7.2 Hz, 2H), 1.16 (s, 12H), 1.15 (s, 3H).

### Step b) Ethyl (E)-3-(3-amino-4-pyridyl)prop-2-enoate

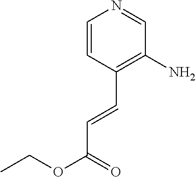

To a solution of 2-ethoxycarbonylvinylboronic acid pinacol ester (9.0 g, 39.8 mmol) in DMF (25 mL) was added 3-amino-4-bromopyridine (6.89 g, 39.81 mmol, CAS RN 239137-39-4), K₂CO₃ (11 g, 79.6 mmol) and 1,1-bis(di-tert-butylphosphino)ferrocene palladium dichloride (2594 mg, 3.98 mmol). The mixture was purged three times with N2. The reaction mixture was heated to 80° C. for 12 h. After cooling to RT the reaction was quenched with water (10 mL), and then evaporated under reduced pressure. The residue was purified by reverse column chromatography (0.1% v/v ammonia in water and MeCN) to provide the desired product as a black solid (2.3 g, 12.0 mmol, 30.1%). MS (ESI): m/z=193.1 [M+H]⁺.

### Step c) 1H-1,7-Naphthyridin-2-one

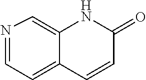 [SMILES: O=c1ccc2ccncc2[nH]1]

To a solution of ethyl (E)-3-(3-amino-4-pyridyl)prop-2-enoate (1800.0 mg, 9.36 mmol) in MeOH (15 mL) was added MeONa (6.94 mL, 37.5 mmol) at 25° C. Then hydroxylamine hydrochloride (2603 mg, 37.46 mmol) was added to mixture, and the mixture was heated to 80° C. for 12 h. The reaction mixture was concentrated and the residue was purified by reverse column chromatography (0.1% v/v FA in water and MeCN) to give the desired compound as yellow solid (1000 mg, 6.84 mmol, 73.1%) which was used in the next step without further purification.

### Step d) 7-Benzyl-1,5,6,8-tetrahydro-1,7-naphthyridin-2-one

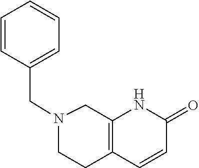 [SMILES: O=c1ccc2c([nH]1)CN(Cc1ccccc1)CC2]

To a solution of 1H-1,7-naphthyridin-2-one (900.0 mg, 6.16 mmol) in EtOH (15 mL) was added benzyl bromide (2106.5 mg, 12.32 mmol and the reaction mixture was stirred at 80° C. for 12 h. Then the mixture was cooled to 0° C. and NaBH₄ (2340 mg, 61.6 mmol) was added carefully. The reaction mixture was poured into 1M HCl aq. (30 mL) and extracted three times with EtOAc (30 mL each). The combined organic layers were dried over Na₂SO₄, filtered and the filtrate evaporated under reduced pressure. The residue was purified by reverse column chromatography (0.1% v/v FA in water and MeCN) to provide the product as a yellow oil (600 mg, 2.5 mmol, 40.6%) which was used in the next step without further purification.

### Step e) 5,6,7,8-Tetrahydro-1H-1,7-naphthyridin-2-one 2,2,2-trifluoroacetic Acid Salt

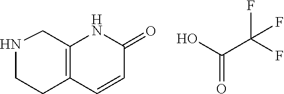

To a solution of 7-benzyl-1,5,6,8-tetrahydro-1,7-naphthyridin-2-one (600.0 mg, 2.5 mmol) in MeOH (10 mL) was added wet Pd/C (60.0 mg, wt. 10%) and TFA (1.0 mL). The reaction mixture was purged with H₂ three times, and then stirred under H₂ atmosphere (balloon) at 25° C. for 4 h. The reaction mixture was filtered, and the filtrate was concentrated in vacuum to provide the crude product which was used in the next step without further purification (500 mg, 1.89 mmol, 75.8%).

### Example 9

### 7-[3-[[2-Fluoro-4-(trifluoromethyl)phenyl]methoxy]azetidine-1-carbonyl]-1,5,6,8-tetrahydro-1,7-naphthyridin-2-one

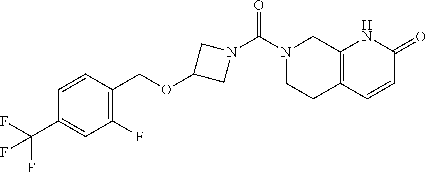 [SMILES: O=C(N1CCc2ccc(=O)[nH]c2C1)N1CC(OCc2ccc(C(F)(F)F)cc2F)C1]

To a solution of (4-nitrophenyl) 3-[[2-fluoro-4-(trifluoromethyl)phenyl]methoxy]azetidine-1-carboxylate (235.23 mg, 0.570 mmol, example 9, step c) and TEA (114.7 mg, 1.14 mmol) in MeCN (5 mL) was added 5,6,7,8-tetrahydro-1H-1,7-naphthyridin-2-one 2,2,2-trifluoroacetic acid salt (100.0 mg, 0.380 mmol, example 8, step e) and the mixture was stirred at 80° C. for 16 h. The mixture was concentrated and the residue purified by prep-HPLC (0.225% v/v FA) and lyophilized to give title compound as a white solid (19.9 mg, 0.050 mmol, 12.2%). MS (ESI): m/z=426.2 [M+H]⁺.

### Step a) tert-Butyl 3-[[2-fluoro-4-(trifluoromethyl)phenyl]methoxy]azetidine-1-carboxylate

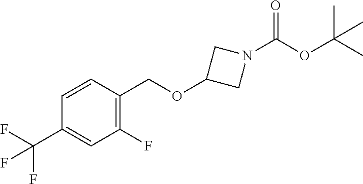 [SMILES: CC(C)(C)OC(=O)N1CC(OCc2ccc(C(F)(F)F)cc2F)C1]

To a solution of [2-fluoro-4-(trifluoromethyl)phenyl]methanol (1500.0 mg, 7.73 mmol CAS RN 197239-49-9) and tert-butyl 3-hydroxyazetidine-1-carboxylate (1405.3 mg, 8.11 mmol, CAS RN 141699-55-0) in toluene (15 mL) was added cyanomethyltributylphosphorane (2797.3 mg, 11.6 mmol) and the mixture was stirred at 100° C. under microwave heating for 1 h. After cooling to RT the mixture was concentrated, and the residue purified by reverse column chromatography (0.1% v/v FA in water and MeCN) to give the title compound (1000 mg, 2.86 mmol, 37.1%) as a light yellow oil. MS (ESI): m/z=294.1 [M-C₄H₈+H]⁺.

### Step b) 3-[[2-Fluoro-4-(trifluoromethyl)phenyl]methoxy]azetidine (2,2,2-trifluoroacetic Acid Salt)

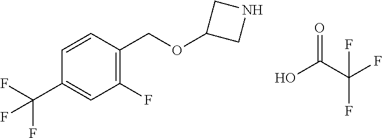 [SMILES: Fc1cc(C(F)(F)F)ccc1COC1CNC1 ; O=C(O)C(F)(F)F]

To a solution of tert-butyl 3-[[2-fluoro-4-(trifluoromethyl)phenyl]methoxy]azetidine-1-carboxylate (2.8 g, 8.02 mmol) in DCM (35 mL) was added TFA (7.0 mL) and the mixture was stirred at 20° C. for 12 h. Evaporation of the reaction mixture gave the title compound (2.9 g, 7.98 mmol, 99.6%) as a light yellow oil. MS (ESI): m/z=250.1 [M+H]⁺.

### Step c) (4-Nitrophenyl) 3-[[2-fluoro-4-(trifluoromethyl)phenyl]methoxy]azetidine-1-carboxylate

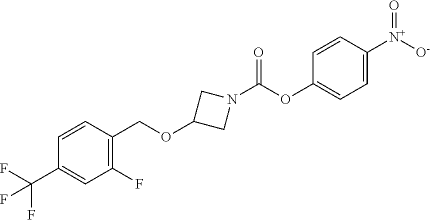 [SMILES: O=C(Oc1ccc([N+](=O)[O-])cc1)N1CC(OCc2ccc(C(F)(F)F)cc2F)C1]

To a solution of 3-[[2-fluoro-4-(trifluoromethyl)phenyl]methoxy]azetidine (2,2,2-trifluoroacetic acid salt) (1.0 g, 2.75 mmol) in DCM (30 mL) was added DIPEA (1065.44 mg, 8.26 mmol) and 4-nitrophenyl chloroformate (554.91 mg, 2.75 mmol) and the reaction mixture was stirred at 25° C. for 12 h. The reaction mixture was washed with water and brine and the organic phase was concentrated in vacuum. The residue was purified by column chromatography (PE:EtOAc=1:0 to 2:1) to give the title compound (900 mg, 2.17 mmol, 78.9%) as a yellow oil. MS (ESI): m/z=415.1 [M+H]⁺.

### Example 10

### rac-(4aS,8aS)-7-(4-Benzhydrylpiperidine-1-carbonyl)octahydro-1,7-naphthyridin-2(1H)-one

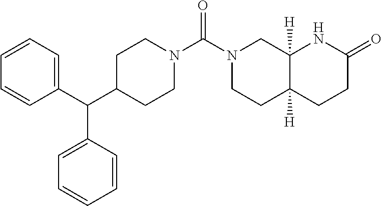 [SMILES: O=C1CC[C@H]2CCN(C(=O)N3CCC(C(c4ccccc4)c4ccccc4)CC3)C[C@H]2N1]

To a mixture of rac-(4aS,8aS)-3,4,4a,5,6,7,8,8a-octahydro-1H-1,7-naphthyridin-2-one (80.0 mg, 0.520 mmol) and DIEA (134.0 mg, 1.04 mmol) in DCM (3 mL) was added 4-benzhydrylpiperidine-1-carbonyl chloride (164.47 mg, 0.520 mmol) and the mixture was stirred at 20° C. for 12 h. The mixture was concentrated and the residue was purified by prep-HPLC (0.5% v/v ammonia in water and MeCN) to give the desired compound as a pink solid (49.3 mg, 0.110 mmol, 22%). MS (ESI): m/z=432.3 [M+H]⁺.

### Step a) Ethyl (E)-3-(4,4,5,5-tetramethyl-1,3,2-dioxaborolan-2-yl)prop-2-enoate

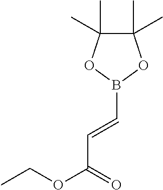 [SMILES: CCOC(=O)/C=C/B1OC(C)(C)C(C)(C)O1]

A solution of copper(I) chloride (158.7 mg, 1.6 mmol), sodium t-butanolate (462.2 mg, 4.81 mmol) and (5-diphenylphosphanyl-9,9-dimethyl-xanthen-4-yl)-diphenyl-phosphane (927.6 mg, 1.6 mmol) in THF (65 mL) was purged with N₂, and stirred at 25° C. for 30 min, then bis(pinacolato)diboron (13.6 g, 53.44 mmol) in THF (33 mL) was added. After stirring for another 10 min, ethyl propiolate (5.4 mL, 53.4 mmol, CAS RN 623-47-2) was added followed by MeOH (4 mL). The reaction mixture was stirred at 25° C. for 11.4 h. After removal of MeOH in vacuum the reaction was quenched with water (50 mL). The mixture was extracted three times with EtOAc (100 mL each), the combined organic layers were washed twice with water (40 mL each) and brine (40 mL), dried over Na₂SO₄ and concentrated. The residue was purified by column chromatography (PE:EA=1:0 to 5:1) to provide the desired compound as a light yellow oil (10.3 g, 45.6 mmol, 85.3%). ¹H NMR (300 MHz, CHLOROFORM-d) δ=6.69-6.60 (m, 1H), 6.55-6.44 (m, 1H), 4.09 (q, J=7.2 Hz, 2H), 1.16 (s, 12H), 1.15 (s, 3H).

### Step b) Ethyl (E)-3-(3-amino-4-pyridyl)prop-2-enoate

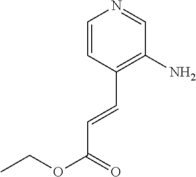

To a solution of 2-ethoxycarbonylvinylboronic acid pinacol ester (9.0 g, 39.8 mmol) in DMF (25 mL) was added 3-amino-4-bromopyridine (6.89 g, 39.8 mmol, CAS RN 239137-39-4), K₂CO₃ (11004 mg, 79.6 mmol) and 1,1-bis(di-tert-butylphosphino)ferrocene palladium dichloride (2595 mg, 3.98 mmol) and the mixture was purged three times with N₂. The reaction mixture was heated to 80° C. for 12 h. The reaction was quenched with water (10 mL), and then the mixture was evaporated. The residue was purified by reverse column chromatography (0.1% v/v ammonia in water and MeCN) to yield the title compound as a black solid (2.3 g, 12.0 mmol, 30.1%). MS (ESI): m/z=193.1 [M+H]⁺.

### Step c) Ethyl 3-(3-aminopyridin-4-yl)propanoate

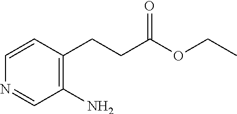

Ethyl (E)-3-(3-amino-4-pyridyl)prop-2-enoate (500.0 mg, 2.6 mmol) in MeOH (10 mL) was added with wet Pd/C (50.0 mg, wt. 10%) at 25° C. The mixture was purged with H2 three times, and then stirred under H₂ atmosphere (balloon) for 12 h. The reaction mixture was filtered and filtrate was concentrated in vacuum to yield the desired compound as a dark brown solid (400 mg, 2.06 mmol, 79.2%).

### Step d) 3,4-Dihydro-1,7-naphthyridin-2(1H)-one

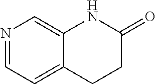 [SMILES: O=C1CCc2ccncc2N1]

Ethyl 3-(3-amino-4-pyridyl)propanoate (250.0 mg, 1.29 mmol) was added to AcOH (5.0 mL, 83.3 mmol) and aq. 11M HCl (6.94 mL) and the mixture was stirred at 90° C. for 12 h. The reaction mixture was diluted with MeOH (2 mL) and directly purified by reverse column chromatography (0.5% v/v ammonia in water and MeCN) to provide the desired compound as a white solid which was pure enough for the next step without further purification. MS (ESI): m/z=147.2 [M+H]⁺.

### Step e) 7-Benzyl-3,4,5,6,7,8-hexahydro-1,7-naphthyridin-2(1H)-one

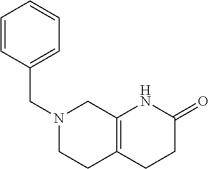 [SMILES: O=C1CCC2=C(CN(Cc3ccccc3)CC2)N1]

To a solution of 3,4-dihydro-1H-1,7-naphthyridin-2-one (200.0 mg, 1.35 mmol) in EtOH (10 mL) was added benzyl bromide (692.6 mg, 4.1 mmol) and the reaction mixture was stirred at 90° C. for 16 h. Then the reaction mixture was cooled to 0° C. and NaBH₄ (513.0 mg, 13.5 mmol) was added carefully. After stirring for 0.5 h, the reaction mixture was poured into sat. aq. NH₄Cl solution (10 mL), extracted with EtOAc (50 mL), the organic layer dried over Na₂SO₄, filtered and concentrated. The residue was purified by reverse column chromatography (0.1% v/v ammonia in water and MeCN) to yield the product as a white solid which was pure enough for the next step without further purification.

### Step f) rac-(4aS,8aS)-3,4,4a,5,6,7,8,8a-Octahydro-1H-1,7-naphthyridin-2-one 2,2,2-trifluoroacetic Acid Salt

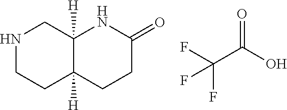

To the solution of 7-benzyl-1,3,4,5,6,8-hexahydro-1,7-naphthyridin-2-one (250.0 mg, 1.03 mmol) in MeOH (5 mL) was added wet Pd/C (50 mg, wt. 10%) and TFA (117.6 mg). The reaction mixture was purged with H2, and then stirred under an atmosphere of H2 (balloon) at 25° C. for 12 h. The reaction mixture was filtered, and the filtrate was concentrated in vacuum to yield the product as a light-yellow oil (100 mg, 0.370 mmol, 62.9%).

### Step g) 4-Benzhydrylpiperidine

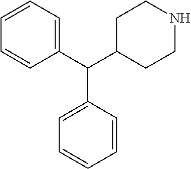 [SMILES: c1ccc(C(c2ccccc2)C2CCNCC2)cc1]

To a mixture of 4-benzhydrylpyridine (5.0 g, 20.4 mmol) in glacial acetic acid (50.0 mL, 20.4 mmol) and was added PtO₂ (462.56 mg, 2.04 mmol) under N₂. The mixture was degassed under vacuum and purged with H₂ 3 times. The reaction mixture was stirred under H2 atmosphere (45 psi) at 85° C. for 12 h. The mixture was filtered and concentrated. The residue was triturated with PE:EtOAc (10:1; 30 mL) and filtered. The filter cake was dried in vacuum to give the desired compound as an off-white solid (4.8 g, 19.1 mmol, 93.7%). MS (ESI): m/z=252.1 [M+H]⁺.

### Step h) 4-Benzhydrylpiperidine-1-carbonyl Chloride

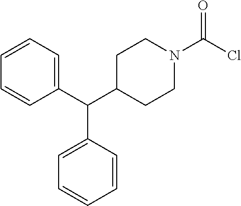 [SMILES: O=C(Cl)N1CCC(C(c2ccccc2)c2ccccc2)CC1]

To a mixture of triphosgene (117.5 mg, 0.400 mmol) and sodium bicarbonate (150.3 mg, 1.79 mmol) in DCM (5 mL) was added a solution of 4-benzhydrylpiperidine (150.0 mg, 0.600 mmol) in DCM (5 mL) dropwise at 0° C. The mixture was stirred at 20° C. for 3 h, filtered and concentrated to give the title compound as light yellow solid (170 mg, 0.540 mmol, 90.8%). MS (ESI) (quenched with MeOH): m/z=310.1 [M-Cl+CH₃OH]⁺.

### Example 11

### rac-(4aS,8aS)-7-[3-[[2-Fluoro-4-(trifluoromethyl)phenyl]methoxy]azetidine-1-carbonyl]-1,3,4,4a,5,6,8,8a-octahydro-1,7-naphthyridin-2-one

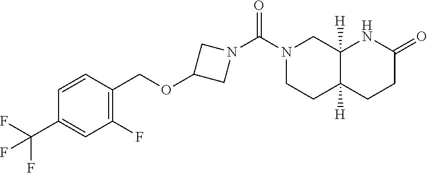 [SMILES: O=C1CC[C@H]2CCN(C(=O)N3CC(OCc4ccc(C(F)(F)F)cc4F)C3)C[C@H]2N1]

To a solution of (4-nitrophenyl) 3-[[2-fluoro-4-(trifluoromethyl)phenyl]methoxy]azetidine-1-carboxylate (162.2 mg, 0.390 mmol, example 9, step c) and TEA (79.07 mg, 0.780 mmol) in MeCN (4 mL) was added 3,4,4a,5,6,7,8,8a-octahydro-1H-1,7-naphthyridin-2-one 2,2,2-trifluoroacetic acid salt (70.0 mg, 0.260 mmol, example 10, step f) and the mixture was stirred at 80° C. for 16 h. The mixture was concentrated, and the residue was purified by prep-HPLC (0.225% v/v FA in water and MeCN) to give the title compound (15.3 mg, 0.040 mmol, 13.3%) as a white solid. MS (ESI): m/z=430.1 [M+H]⁺.

### Example 12

### rac-(4aR,8aS)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one

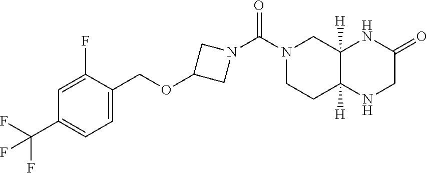 [SMILES: O=C1CN[C@H]2CCN(C(=O)N3CC(OCc4ccc(C(F)(F)F)cc4F)C3)C[C@H]2N1]

To a solution of tert-butyl (4aR,8a5)-6-[3-[[2-fluoro-4-(trifluoromethyl)phenyl]methoxy]azetidine-1-carbonyl]-3-oxo-4,4a,5,7,8,8a-hexahydro-2H-pyrido[3,4-b]pyrazine-1-carboxylate (30.0 mg, 0.060 mmol) in DCM (2 mL) was added TFA (0.4 mL) and the mixture was stirred at 25° C. for 2 h. The mixture was concentrated and the residue was purified by prep-HPLC (0.225% v/v FA in water and MeCN) to give the desired product (2.5 mg, 0.010 mmol, 10.2%, 99.5% purity) as a white solid. MS (ESI): m/z=431.3 [M+H]⁺.

### Step a) Methyl 2-[(3-nitro-4-pyridyl)amino]acetate

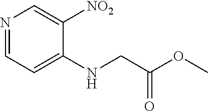

A mixture of 4-chloro-3-nitropyridine (5.0 g, 31.5 mmol, CAS RN 13091-23-1), glycine methyl ester hydrochloride (5.94 g, 47.3 mmol, CAS RN 5680-79-5) and TEA (13.2 mL, 94.6 mmol) in 1,4-dioxane (75 mL) was stirred at 25° C. for 12 h. Then the mixture was diluted with water (100 mL) and extracted three times with EtOAc (150 mL each). The combined organic layers were dried over Na₂SO₄, filtered and the filtrate evaporated. The residue was purified by silica gel column chromatography (PE:EtOAc=1:1) to give the desired product (5185 mg, 24.6 mmol, 77.9%) as a yellow solid.

### Step b) 2,4-Dihydro-1H-pyrido[3,4-b]pyrazin-3-one

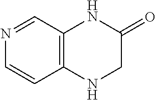 [SMILES: O=C1CNc2ccncc2N1]

To a solution of methyl 2-[(3-nitro-4-pyridyl)amino]acetate (2000.0 mg, 9.47 mmol) in MeOH (80 mL) was added wet Pd/C (400.0 mg, wt. 10%) and the mixture was stirred at 25° C. under H₂ atmosphere (balloon) for 12 h. The mixture was filtered, the filter cake was washed with DCM:MeOH (3:1, 200 mL) and the combined filtrates were evaporated to give the desired product (1000 mg, 6.7 mmol, 70.8%) as yellow solid. MS (ESI): m/z=150.1 to [M+H]⁺.

### Step c) tert-Butyl 3-oxo-2,4-dihydropyrido[3,4-b]pyrazine-1-carboxylate

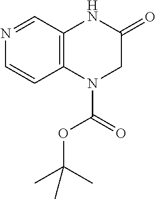 [SMILES: CC(C)(C)OC(=O)N1CC(=O)Nc2cnccc21]

Di-tert-butyl dicarbonate (2195 g, 10.1 mmol) was added to a solution of 2,4-dihydro-1H-pyrido[3,4-b]pyrazin-3-one (1000 mg, 6.7 mmol), TEA (1.9 mL, 13.4 mmol) and DMAP (163.8 mg, 1.34 mmol) in DMF (50 mL) at 0° C., and the reaction mixture was stirred at 25° C. for 12 h. The mixture was diluted with water (200 mL) and extracted three times with EtOAc (50 mL each). The combined organic phase was washed with brine, dried over Na₂SO₄ and concentrated. The residue was purified by silica gel column chromatography (PE:EtOAc=5:1 to 0:1) to give the desired product (1000 mg, 4.01 mmol, 59.8%) as a light yellow solid. MS (ESI): m/z=194.0 [M-C₄H₈+H]⁺.

### Step d) tert-Butyl 6-benzyl-3-oxo-2,4-dihydropyrido[3,4-b]pyrazin-6-ium-1-carboxylate Bromide

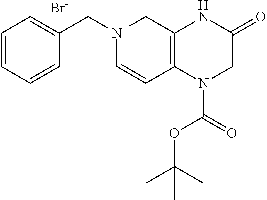 [SMILES: [Br-] ; CC(C)(C)OC(=O)N1CC(=O)NC2=C1C=C[NH+](Cc1ccccc1)C2]

To a solution of tert-butyl 3-oxo-2,4-dihydropyrido[3,4-b]pyrazine-1-carboxylate (800.0 mg, 3.21 mmol) in DCM (30 mL) was added benzyl bromide (0.76 mL, 6.42 mmol) and the mixture was stirred at 20° C. for 12 h. Then the mixture was filtered and the filter cake washed with DCM (5 mL). Light yellow solid (900 mg, 2.14 mmol, 66.7%). MS (ESI): m/z=284.3 [M-C₄H₈-Br+H]⁺.

### Step e) tert-Butyl 6-benzyl-3-oxo-4,5,7,8-tetrahydro-2H-pyrido[3,4-b]pyrazine-1-carboxylate

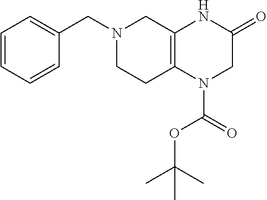 [SMILES: CC(C)(C)OC(=O)N1CC(=O)NC2=C1CCN(Cc1ccccc1)C2]

To a solution of tert-butyl 6-benzyl-3-oxo-2,4-dihydropyrido[3,4-b]pyrazin-6-ium-1-carboxylate bromide (850.0 mg, 2.02 mmol) in MeOH (30 mL) was added NaBH₄ (765.1 mg, 20.2 mmol) portionwise at 0° C. and the mixture was stirred at 0° C. for 2 h. Another batch of NaBH₄ (229.5 mg, 6.07 mmol) was added portionwise at 0° C. and stirring was continued at 0° C. for another 2 h. Then the mixture was poured into aq. NH₄Cl solution (50 mL) and extracted three times with EtOAc (30 mL each), the combined organic phase was washed with brine, dried over Na₂SO₄ and concentrated to give desired product as a light yellow solid (500 mg, 1.5 mmol, 72%). MS (ESI): m/z=344.3 [M+H]⁺.

### Step f) tert-Butyl 3-oxo-2,4,5,6,7,8-hexahydropyrido[3,4-b]pyrazine-1-carboxylate

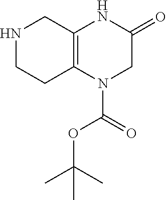 [SMILES: CC(C)(C)OC(=O)N1CC(=O)NC2=C1CCNC2]

To a solution of tert-butyl 6-benzyl-3-oxo-4,5,7,8-tetrahydro-2H-pyrido[3,4-b]pyrazine-1-carboxylate (100.0 mg, 0.290 mmol) in MeOH (10 mL) and ammonia (0.05 mL) was added wet Pd/C (20.0 mg, wt. 10%) and the mixture was stirred at 20° C. under H2 atmosphere (balloon) for 12 h. The reaction mixture was filtered and the filtrate was concentrated. The residue was dissolved in EtOAc (10 mL), and another batch wet Pd/C (20.0 mg, wt. 10%) was added and the mixture was stirred at 20° C. under H2 atmosphere (balloon) for another 24 h. Filtration and evaporation of the filtrate gave the desired product as a colorless oil (70 mg, 0.28 mmol, 94.9%). MS (ESI): m/z=507.2 [2M+H]⁺.

### Step g) tert-Butyl 6-[3-[[2-fluoro-4-(trifluoromethyl)phenyl]methoxy]azetidine-1-carbonyl]-3-oxo-4,5,7,8-tetrahydro-2H-pyrido[3,4-b]pyrazine-1-carboxylate

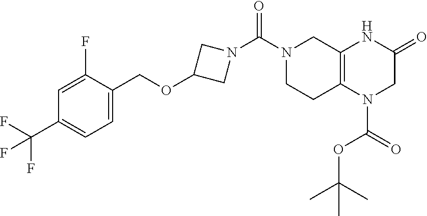 [SMILES: CC(C)(C)OC(=O)N1CC(=O)NC2=C1CCN(C(=O)N1CC(OCc3ccc(C(F)(F)F)cc3F)C1)C2]

A solution of tert-butyl 3-oxo-2,4,5,6,7,8-hexahydropyrido[3,4-b]pyrazine-1-carboxylate (70.0 mg, 0.280 mmol), (4-nitrophenyl) 3-[[2-fluoro-4-(trifluoromethyl)phenyl]methoxy]azetidine-1-carboxylate (114.5 mg, 0.280 mmol) and TEA (0.12 mL, 0.830 mmol) in ACN (2 mL) was stirred at 80° C. for 16 h. The mixture was concentrated and the residue was purified by reverse flash chromatography (0.1% v/v FA in water and MeCN) to give the desired product as light yellow oil (40 mg, 0.080 mmol, 27.4%). MS (ESI): m/z=473.2 [M-C₄H₈+H]⁺.

### Step h) tert-Butyl rac-(4aR,8aS)-6-[3-[[2-fluoro-4-(trifluoromethyl)phenyl]methoxy]azetidine-1-carbonyl]-3-oxo-4,4a,5,7,8,8a-hexahydro-2H-pyrido[3,4-b]pyrazine-1-carboxylate

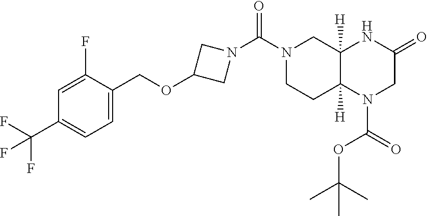 [SMILES: CC(C)(C)OC(=O)N1CC(=O)N[C@@H]2CN(C(=O)N3CC(OCc4ccc(C(F)(F)F)cc4F)C3)CC[C@@H]21]

To a solution of tert-butyl 6-[3-[[2-fluoro-4-(trifluoromethyl)phenyl]methoxy]azetidine-1-carbonyl]-3-oxo-4,5,7,8-tetrahydro-2H-pyrido[3,4-b]pyrazine-1-carboxylate (40.0 mg, 0.080 mmol) in MeOH (5 mL) was added wet Pd/C (10.0 mg, wt. 10%) and then the mixture was stirred at 30° C. under H2 atmosphere (balloon) for 24 h. The mixture was filtered and the filtrate was concentrated to provide the crude product (30 mg, 0.06 mmol, 74.7%) as a colorless oil. MS (ESI): m/z=531.2 [M+H]⁺.

### Example 13 and Example 14

### (4aR,8aS)- or (4aR,8aS)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one and (4aS,8aR)- or (4aS,8aR)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one

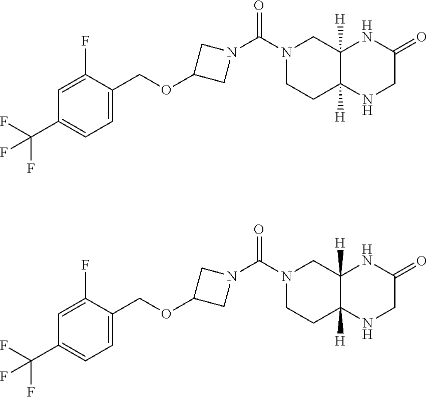 [SMILES: O=C1CN[C@H]2CCN(C(=O)N3CC(OCc4ccc(C(F)(F)F)cc4F)C3)C[C@H]2N1 ; O=C1CN[C@@H]2CCN(C(=O)N3CC(OCc4ccc(C(F)(F)F)cc4F)C3)C[C@@H]2N1]

The enantiomers of example 12 were separated by preparative chiral-HPLC (DAICEL CHIRALCEL® OD (250 mm*30 mm, 10 μm)), eluant: 30% EtOH ((containing 0.1% ammonia) in supercritical CO₂) to give the desired enantiomers as light yellow oils.

Enantiomer A (first eluting enantiomer): MS (ESI): m/z=431.2 [M+H]⁺.

Enantiomer B (second eluting enantiomer): MS (ESI): m/z=431.1 [M+H]⁺.

### Example 15

### 6-[3-[[2-Fluoro-4-(trifluoromethyl)phenyl]methoxy]azetidine-1-carbonyl]-1,2,4,5,7,8-hexahydropyrido[3,4-b]pyrazin-3-one

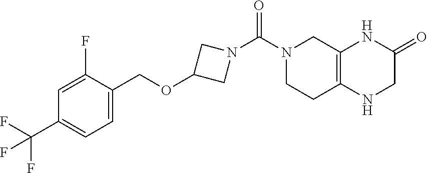 [SMILES: O=C1CNC2=C(CN(C(=O)N3CC(OCc4ccc(C(F)(F)F)cc4F)C3)CC2)N1]

To a solution of tert-butyl 6-[3-[[2-fluoro-4-(trifluoromethyl)phenyl]methoxy]azetidine-1-carbonyl]-3-oxo-4,5,7,8-tetrahydro-2H-pyrido[3,4-b]pyrazine-1-carboxylate (20.0 mg, 0.040 mmol, example 12, step g) in DCM (1 mL) was added TFA (0.2 mL) and the mixture was stirred at 20° C. for 12 h. The mixture was concentrated and the residue was purified by prep-HPLC (0.225% v/v FA in water and MeCN) to give the desired product as light yellow solid (1 mg, 5.7%). MS (ESI): m/z=429.2 [M+H]⁺.

### Example 16

### (4aS,8aS)-6-[3-[[2-fluoro-4-(trifluoromethyl)phenyl]methoxy]azetidine-1-carbonyl]-4a-hydroxy-5,7,8,8a-tetrahydro-4H-pyrido[4,3-b][1,4]oxazin-3-one

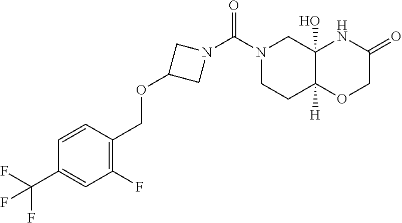 [SMILES: O=C1CO[C@H]2CCN(C(=O)N3CC(OCc4ccc(C(F)(F)F)cc4F)C3)C[C@@]2(O)N1]

Biotransformation with E. coli-expressed CYP3A4 (intact cells OD 10.0). Reaction Composition: In 100 ml 0.1 M K⁺ phosphate buffer pH 7.4, 27 deg, 200 rpm in 100 ml glass baffled flask. (4aR,8a5)-6-(3-((2-fluoro-4-(trifluoromethyl)benzyl)oxy)azetidine-1-carbonyl)hexahydro-2H-pyrido[4,3-b][1,4]oxazin-3(4H)-one was added to final concentration of 0.1 mM. After 3 h incubation a further 3 ml aliquot of washed cell susp. and 0.5 ml of 0.1 mM NADP was added and the incubation continued for a further 2 h. The broth was then centrifuged and the supernatant applied to a 10 g C18 cartridge which was eluted with a step gradient of acetonitrile in water. After lyophilization purification on analytical HPLC; XDB C18, 150×4.6 mm, gradient of MeCN in 0.05% TFA/H₂O. Product was obtained as lyophilized powder. MS (ESI): m/z=476.4 [M+H]⁺

### Example 17

### 6-[3-[[2-fluoro-4-(trifluoromethyl)phenyl]methoxy]azetidine-1-carbonyl]-4,5,7,8-tetrahydropyrido[4,3-b][1,4]thiazin-3-one

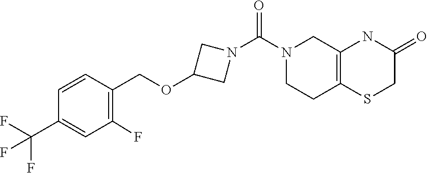 [SMILES: O=C1CSC2=C(CN(C(=O)N3CC(OCc4ccc(C(F)(F)F)cc4F)C3)CC2)N1]

5,6,7,8-tetrahydro-2H-pyrido[4,3-b][1,4]thiazin-3(4H)-one hydrochloride (22 mg, 106 μmol) was dissolved in acetonitrile (1 ml) and TEA (75.4 mg, 104 μl, 745 μmol7) was added. Then 1,1′-carbonyl-di(1,2,4-triazole) (17.5 mg, 106 μmol) was added and the reaction was stirred for 40 min to form the active ester. Then 3-((2-fluoro-4-(trifluoromethyl)benzyl)oxy)azetidine 4-methylbenzenesulfonate (53.8 mg, 128 μmol) was added and stirred for 2 hr at 25° C. The reaction mixture was quenched with 2 ml water and extracted with 2×20 ml ethyl acetate, 2×10 ml 5% NaHCO₃, 5 ml 0.5N HCl, brine, dried over MgSO₄, and solvent was removed under vacuum. The crude was purified by prep. HPLC Gemini NX, 12 nm, 5 μm, 100×30 mm, ACN/Water+0.1% HCOOH, the pooled fractions were lyophilized to get the product (37 mg, 74%) as a light yellow, lyophilized solid. MS (ESI): m/z=446.3 [M+H]⁺

### Step a) 6-benzyl-3-oxo-3,4-dihydro-2H-pyrido[4,3-b][1,4]thiazin-6-ium Bromide

To a capped vial was added 2H-pyrido[4,3-b][1,4]thiazin-3(4H)-one (750 mg, 4.51 mmol), followed by DCM (7.8 ml) to form a suspension. A solution of (bromomethyl)benzene (926 mg, 644 μl, 5.42 mmol) in MeOH (1.95 ml) was added and the suspension was stirred at RT for 4 days. The suspension was cooled down to 4° C. and then filtered. The off-white solid was washed three times with DCM/n-hexane (1:3) and dried to obtain 6-benzyl-3-oxo-3,4-dihydro-2H-pyrido[4,3-b][1,4]thiazin-6-ium bromide (1.32 g, 86.7% yield) as an off-white solid. MS (ESI): m/z=257.2 [M−H-Br]⁺

### Step b) 6-benzyl-5,6,7,8-tetrahydro-2H-pyrido[4,3-b][1,4]thiazin-3(4H)-one

To a solution of 6-benzyl-3-oxo-3,4-dihydro-2H-pyrido[4,3-b][1,4]thiazin-6-ium bromide (500 mg, 1.48 mmol) in Methanol (20 ml) was added in portions sodium borohydride (67.3 mg, 1.78 mmol) at 20° C. (gas evolution). The yellow solution was stirred at 20° C. for 1 h. The reaction mixture was quenched with 0.5 ml water and 0.5 ml sat. NH₄Cl, solvent was removed in vacuo. The residue was extracted with EtOAc, water and brine, dried with MgSO₄, solvent was removed in vacuo. The crude residue (390 mg) was purified by prep HPLC, Gemini NX, 12 nm, 5 μm, 100×30 mm, ACN/Water+0.1% TEA, the collected fractions were lyophilized, to get the expected product (70 mg, 18%) MS (ESI): m/z=261.1 [M+H]⁺

### Step c) 5,6,7,8-tetrahydro-2H-pyrido[4,3-b][1,4]thiazin-3(4H)-one Hydrochloride

To a solution of 6-benzyl-5,6,7,8-tetrahydro-2H-pyrido[4,3-b][1,4]thiazin-3(4H)-one (60 mg, 230 μmol) in DCM (2 ml) at 0-4° C. was added 1-chloroethyl chloroformate (39.5 mg, 30 μl, 277 μmol) and stirred 10 min, then 10 min at 5-20° C., solvent was removed in vacuo. The residue was dissolved again in methanol (2 ml) and heated at 75° C. for 40 min. The yellow solution was concentrated, the residue was dissolved in 1 ml of MeOH and the product was precipitated with diethyl ether at 20° C., the organic phase was decanted off twice and washed with diethyl ether. The desired product was obtained as a light yellow solid 22 mg (42%) MS (ESI): m/z=171.1 [M+H]⁺

### Example 18

### rac-(4aS,8aS)-7-[3-[[2-fluoro-4-(trifluoromethyl)phenyl]methoxy]azetidine-1-carbonyl]-4-hydroxy-1,3,4,4a,5,6,8,8a-octahydro-1,7-naphthyridin-2-one

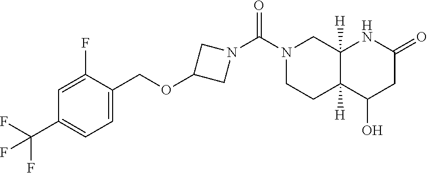 [SMILES: O=C1CC(O)[C@H]2CCN(C(=O)N3CC(OCc4ccc(C(F)(F)F)cc4F)C3)C[C@H]2N1]

To a solution of (4aS,8aS)-4-[tert-butyl(diphenyl)silyl]oxy-7-[3-[[2-fluoro-4-(trifluoromethyl)phenyl]methoxy]azetidine-1-carbonyl]-1,3,4,4a,5,6,8,8a-octahydro-1,7-naphthyridin-2-one (20.0 mg, 0.030 mmol) in methanol (2 mL), ammonium fluoride (21.67 mg, 0.580 mmol) was added and stirred at 50° C. for 12 h. LCMS showed the reactant was consumed and the desired mass of the target product was detected, the reaction mixture was filtered and the filtrate was purified with Prep-HPLC (0.225% v/v formic acid) and lyophilized to give (4aS,8a5)-7-[3-[[2-fluoro-4-(trifluoromethyl)phenyl]methoxy]azetidine-1-carbonyl]-4-hydroxy-1,3,4,4a,5,6,8,8a-octahydro-1,7-naphthyridin-2-one (2.8 mg, 0.010 mmol, 21% yield) as white solid. MS (ESI): m/z=446.2 [M+H]⁺

### Step a) tert-butyl N-(4-methyl-3-pyridyl)carbamate

To a flame-dried 500 mL round-bottom flask purged with N2 was added 3-amino-4-methylpyridine (15.0 g, 138.71 mmol) and THF (150 mL), NaHMDS (166.0 mL, 166 mmol) was added dropwise at 0° C. over 1 h and the resulting red solution was stirred for 30 min. Di-t-butyldicarbonate (34.47 mL, 152.58 mmol, 1.1 eq) was added dropwise over 5 min, then the mixture was stirred at 25 C.° for 12 hr. TLC showed the reactant was partly consumed and new spots were detected, the residue was taken up in water (200 ml) and washed by EtOAc (100 ml, three times), then poured 50 ml brine in the organics. The organics were then separated and dried (MgSO₄) before concentration to dryness. The crude was then purified by silica gel column (Pentane:EA=10:1 to 3:1) to give tert-butyl N-(4-methyl-3-pyridyl)carbamate (6 g, 20.8% yield) as yellow oil and tert-butyl N-tert-butoxycarbonyl-N-(4-methyl-3-pyridyl)carbamate (10 g, 23.4% yield) as yellow solid.

### Step b) tert-butyl N-(4-formyl-3-pyridyl)carbamate

To a solution of tert-butyl N-(4-methyl-3-pyridyl)carbamate (5000 mg, 24.01 mmol) in 1,4-Dioxane (50 mL), SeO₂ (4030 mg, 36.01 mmol) was added and stirred at 105° C. for 3 h. TLC (Pentane/EA=1/1) showed the reactant was partly consumed and new spots were detected. The reaction mixture was filtered and the filtrate was purified with silica column chromatography (Pentane/EA=20/1 to 3/1) to give tert-butyl N-(4-formyl-3-pyridyl)carbamate (1800 mg, 8.1 mmol, 33.74% yield) as yellow oil.

### Step c) ethyl 3-[3-(tert-butoxycarbonylamino)-4-pyridyl]-3-hydroxy-propanoate

A solution of tert-butyl N-(4-formyl-3-pyridyl)carbamate (2700 mg, 12.15 mmol) and ethyl (trimethylsilyl)acetate (2434.21 mg, 15.19 mmol) in THF (54 mL) was treated with Tetrabutylammonium acetate (366.05 mg, 1.21 mmol), the result solution was stirred at 25° C. for 2 h, Aqueous HCl (2N, 5 mL) was added and stirred for 30 min, LCMS showed the reactant was consumed completely and the desired mass of the target product was detected, the reaction mixture was neutralized with saturated aqueous NaHCO₃ solution. extracted with EtOAc (50 mL*3), the organic layer was purified with silica column chromatography (PE/EA=20/1 to 3/1) to give ethyl 3-[3-(tert-butoxycarbonylamino)-4-pyridyl]-3-hydroxy-propanoate (3100 mg, 9.99 mmol, 85.38% yield) as yellow oil. MS (ESI): m/z=311.1 [M+H]+ (biggest peak sufficient)

### Step d) ethyl 3-[3-(tert-butoxycarbonylamino)-4-pyridyl]-3-[tert-butyl(diphenyl)silyl]oxy-propanoate

To a solution of ethyl 3-[3-(tert-butoxycarbonylamino)-4-pyridyl]-3-hydroxy-propanoate (3100.0 mg, 9.05 mmol, 1 eq) and imidazole (1232.6 mg, 18.11 mmol, 2 eq) in DCM (75 mL), TBDPSCl (3722 mg, 13.58 mmol, 1.5 eq) was added and stirred at 20° C. for 12 h. LCMS showed the reactant was consumed and the desired mass of the target product was detected, the reaction mixture was poured into H₂O (20 mL) and extracted with DCM (20 mL*3), the organic layer was evaporated under reduced pressure to give the crude, which was then purified with MPLC (Pentane/EA=3/1) and evaporated to give ethyl 3-[3-(tert-butoxycarbonylamino)-4-pyridyl]-3-[tert-butyl(diphenyl)silyl]oxy-propanoate (4700 mg, 94.6% yield) as a colorless oil. MS (ESI): m/z=549.2 [M+H]⁺

### Step e) 4-[tert-butyl(diphenyl)silyl]oxy-3,4-dihydro-1H-1,7-naphthyridin-2-one

To a solution of ethyl 3-[3-(tert-butoxycarbonylamino)-4-pyridyl]-3-[tert-butyl(diphenyl)silyl]oxy-propanoate (1900 mg, 3.46 mmol) in DCM (20 mL) was added TFA (4.0 mL, 3.46 mmol), the mixture was stirred at 20° C. for 12 h, LCMS showed the reactant was consumed completely and the desired mass of the target product was detected. TEA was added to the reaction mixture slowly till PH>7 and then evaporated under reduced pressure to give the crude, which was then purified with reversed phase column (NH3.H2O) and lyophilized to give 4-[tert-butyl(diphenyl)silyl]oxy-3,4-dihydro-1H-1,7-naphthyridin-2-one (800 mg, 57.4% yield) as a white solid. MS (ESI): m/z=403.1 [M+H]⁺

### Step f) 7-benzyl-4-[tert-butyl(diphenyl)silyl]oxy-3,4-dihydro-1H-1,7-naphthyridin-7-ium-2-one Bromide

To a solution of 4-[tert-butyl(diphenyl)silyl]oxy-3,4-dihydro-1H-1,7-naphthyridin-2-one (800.0 mg, 1.99 mmol) in DCM (12 mL) was added another solution benzyl bromide (0.71 mL, 5.96 mmol) in DCM, the mixture was stirred at 20° C. for 12 h, LCMS showed the reactant was consumed completely and the desired mass of the target product was detected, after reaction finished, white solid was observed, the reaction mixture was filtered and washed with MTBE (20 mL), the filter cake 7-benzyl-4-[tert-butyl(diphenyl)silyl]oxy-3,4-dihydro-1H-1,7-naphthyridin-7-ium-2-one bromide (1600 mg, 2.79 mmol, 140.36% yield) was collected as a white solid. MS (ESI): m/z=493.1 [M+H]⁺

### Step g) 7-benzyl-4-[tert-butyl(diphenyl)silyl]oxy-1,3,4,5,6,8-hexahydro-1,7-naphthyridin-2-one

A solution of 7-benzyl-4-[tert-butyl(diphenyl)silyl]oxy-3,4-dihydro-1H-1,7-naphthyridin-7-ium-2-one bromide (1600 mg, 2.79 mmol) in methanol (27.54 mL) was stirred at 0° C., NaBH₄ (2120 mg, 55.79 mmol) was added in batches, the mixture was allowed to warm to room temperature and stirred at 20° C. for 12 h, LCMS showed the reactant was consumed completely and the desired mass of the target product was detected. The reaction mixture was added saturated NH₄Cl solution slowly and then added H₂O (5 mL), then extracted with EtOAc (5 mL*3), the organic layer was evaporated under reduced pressure (25° C.) to give the crude, which was purified with reversed phase column (FA 0.25%) and lyophilized to give 7-benzyl-4-[tert-butyl(diphenyl)silyl]oxy-1,3,4,5,6,8-hexahydro-1,7-naphthyridin-2-one (600 mg, 37.27% yield) as white solid. MS (ESI): m/z=497.3 [M+H]⁺

### Step h): tert-butyl (4aS,8aS)-4-[tert-butyl(diphenyl)silyl]oxy-2-oxo-1,3,4,4a,5,6,8,8a-octahydro-1,7-naphthyridine-7-carboxylate

A solution of 7-benzyl-4-[tert-butyl(diphenyl)silyl]oxy-1,3,4,5,6,8-hexahydro-1,7-naphthyridin-2-one (150.0 mg, 0.300 mmol), di-tert-butyl dicarbonate (90 mg, 0.4 mmol) and wet Pd/C (200.0 mg, 0.300 mmol) in Methanol (9 mL) was purged with H2 for 3 times, then stirred at 25° C. for 24 h. LCMS showed the reactant was consumed completely and the desired mass of the target product was detected, the reaction mixture was filtered and the filtrate was evaporated under reduced pressure to give tert-butyl 4-[tert-butyl(diphenyl)silyl]oxy-2-oxo-1,3,4,4a,5,6,8,8a-octahydro-1,7-naphthyridine-7-carboxylate (150 mg, 0.290 mmol, 97.64% yield) as a yellow oil. MS (ESI): m/z=453.1 [M−56+H]⁺

### Step i): (4aS,8aS)-4-[tert-butyl(diphenyl)silyl]oxy-3,4,4a,5,6,7,8,8a-octahydro-1H-1,7-naphthyridin-2-one

A mixture of TFA (1.0 mL, 6.37 mmol), tert-butyl (4aS,8aS)-4-[tert-butyl(diphenyl)silyl]oxy-2-oxo-1,3,4,4a,5,6,8,8a-octahydro-1,7-naphthyridine-7-carboxylate (150.0 mg, 0.290 mmol) in DCM (25 mL) was stirred at 25° C. for 12 h. TLC showed the reactant was consumed completely and a new spot was detected, TEA was added to the reaction mixture until PH>8, then H₂O was added to the solution and extracted with DCM (10 mL*3), the organic layer was evaporated under reduced pressure (25° C.) to give (4aS,8aS)-4-[tert-butyl(diphenyl)silyl]oxy-3,4,4a,5,6,7,8,8a-octahydro-1H-1,7-naphthyridin-2-one (30 mg, 0.070 mmol, 24.9% yield) as a yellow oil.

### Step j): (4-nitrophenyl) (4a5,8a5)-4-[tert-butyl(diphenyl)silyl]oxy-2-oxo-1,3,4,4a,5,6,8,8a-octahydro-1,7-naphthyridine-7-carboxylate

To a solution of (4aS,8aS)-4-[tert-butyl(diphenyl)silyl]oxy-3,4,4a,5,6,7,8,8a-octahydro-1H-1,7-naphthyridin-2-one (30.0 mg, 0.070 mmol) in DCM (1 mL) was added DIPEA (23.71 mg, 0.180 mmol), the temperature was kept at 0° C., then 4-nitrophenyl chloroformate (16.28 mg, 0.080 mmol) was added. The reaction mixture was stirred at 25° C. for 12 h. LCMS showed that desired product was detected, the reaction mixture was evaporated under reduced pressure to give the crude, which was then purified with prep-HPLC (0.225% v/v FA) and lyophilized to give (4-nitrophenyl) (4a5,8a5)-4-[tert-butyl(diphenyl)silyl]oxy-2-oxo-1,3,4,4a,5,6,8,8a-octahydro-1,7-naphthyridine-7-carboxylate (20 mg, 47.48% yield) as white solid. MS (ESI): m/z=574.1 [M+H]⁺

### Step k) (4a5,8a5)-4-[tert-butyl(diphenyl)silyl]oxy-7-[3-[[2-fluoro-4-(trifluoromethyl)phenyl]methoxy]azetidine-1-carbonyl]-1,3,4,4a,5,6,8,8a-octahydro-1,7-naphthyridin-2-one

A solution of 3-[[2-fluoro-4-(trifluoromethyl)phenyl]methoxy]azetidine trifluoroacetate (15.19 mg, 0.040 mmol) and DIEA (0.2 mL, 0.030 mmol) in ACN (1 mL), (4-nitrophenyl) (4a5,8a5)-4-[tert-butyl(diphenyl)silyl]oxy-2-oxo-1,3,4,4a,5,6,8,8a-octahydro-1,7-naphthyridine-7-carboxylate (20.0 mg, 0.030 mmol) was added and stirred at 80° C. for 12 h. TLC showed the reactant was consumed completely and the desired mass of the target product was detected, the reaction mixture was poured into H₂O (5 mL), extracted with EtOAc (5 mL*3), the organic layer was purified with silica column chromatography (Pentane/EA=10/1 to pure EA) to give (4aS,8a5)-4-[tert-butyl(diphenyl)silyl]oxy-7-[3-[[2-fluoro-4-(trifluoromethyl)phenyl]methoxy]azetidine-1-carbonyl]-1,3,4,4a,5,6,8,8a-octahydro-1,7-naphthyridin-2-one (20 mg, 83.9% yield) as yellow solid.

### Example 19

A compound of formula (I) can be used in a manner known per se as the active ingredient for the production of tablets of the following composition:

Per tablet

| | |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example 20

A compound of formula (I) can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1-34. (canceled)

35. A compound of formula (I): 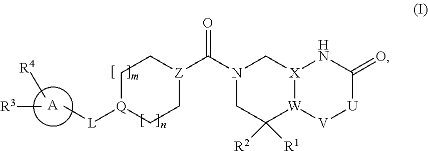
or a pharmaceutically acceptable salt thereof, wherein:
(i) U is CH₂;
V is O;
W and X are both CH;
R¹ is halogen or C₁₋₆-alkyl; and
R² is hydrogen, halogen, or C₁₋₆-alkyl; or
R¹ and R², taken together with the carbon atom to which they are attached, form a C₃-C₁₀-cycloalkyl; or
(ii) U is CH₂;
V is O;
W is CR^{w};
X is CH;
R^{w} is halogen or C₁₋₆-alkyl;
R¹ and R² are independently selected from the group consisting of hydrogen, halogen, and C₁₋₆-alkyl; or
R¹ and R², taken together with the carbon atom to which they are attached, form a C₃-C₁₀-cycloalkyl; or
(iii) U is CH₂;
V is O;
W and X together form a group C═C; and
R¹ and R² are independently selected from the group consisting of hydrogen, halogen, and C₁₋₆-alkyl; or
R¹ and R², taken together with the carbon atom to which they are attached, form a C₃-C₁₀-cycloalkyl; or
(iv) U is CH₂;
V is NH, CH₂, S, S═O, SO₂, CHOH, CHF, or CF₂;
(a) W is CR^{w}, and
X is CH; or
(b) W and X together form a group C═C;
R^{w} is hydrogen, halogen, or C₁₋₆-alkyl;
R¹ and R² are independently selected from the group consisting of hydrogen, halogen, and C₁₋₆-alkyl; or
R¹ and R², taken together with the carbon atom to which they are attached, form a C₃-C₁₀-cycloalkyl; or
(v) U and V together form a group C═C;
W and X together form a group C═C; and
R¹ and R² are independently selected from the group consisting of hydrogen, halogen, and C₁₋₆-alkyl; or
R¹ and R², taken together with the carbon atom to which they are attached, form a C₃-C₁₀-cycloalkyl; or
(vi) U is CH₂;
V is O;
W is CH;
X is C—OH; and
R¹ and R² are independently selected from the group consisting of hydrogen, halogen, and C₁₋₆-alkyl; or
R¹ and R², taken together with the carbon atom to which they are attached, form a C₃-C₁₀-cycloalkyl;
m and n are both 0; or
m and n are both 1;
Z is CH or N;
Q is CR^{q} or N;
R^{q} is hydrogen, halogen, hydroxy, halo-C₁₋₆-alkyl, or C₁₋₆-alkyl;
L is a covalent bond, —CHR⁵—, —O—, —OCH₂—, —CH₂O—, —CH₂OCH₂—, —CF₂CH₂—, or —CH₂CF₂—;
A is C₆-C₁₄-aryl, 5- to 14-membered heteroaryl, or 3- to 14-membered heterocyclyl;
R³ and R⁴ are independently selected from the group consisting of hydrogen, halogen, SF₅, cyano, C₁₋₆-alkyl, C₁₋₆-alkoxy, halo-C₁₋₆-alkyl, halo-C₁₋₆-alkoxy, C₆-C₁₄-aryl, C₃-C₁₀-cycloalkyl, 5-14-membered heteroaryl, C₆-C₁₄-aryloxy, C₃-C₁₀-cycloalkyloxy, and 5-14-membered heteroaryloxy,
wherein said C₆-C₁₄-aryl, C₃-C₁₀-cycloalkyl, 5-14-membered heteroaryl, C₆-C₁₄-aryloxy, C₃-C₁₀-cycloalkyloxy, and 5-14-membered heteroaryloxy, are optionally substituted with 1-2 substituents selected from the group consisting of halogen, C₁₋₆-alkyl, and halo-C₁₋₆-alkyl; and
R⁵ is hydrogen or C₆-C₁₄-aryl.

36. The compound of claim 35, or a pharmaceutically acceptable salt thereof, wherein:
(i) U is CH₂;
V is O;
W and X are both CH;
R¹ is halogen or C₁₋₆-alkyl; and
R² is hydrogen or halogen; or
(ii) U is CH₂;
V is O;
W and X together form a group C═C; and
R¹ and R² are both hydrogen; or
(iii) U is CH₂;
V is NH, S, or CH₂;
(a) W and X are both CH; or
(b) W and X together form a group C═C; and
R¹ and R² are both hydrogen;
(iv) U and V together form a group C═C;
W and X together form a group C═C; and
R¹ and R² are both hydrogen; or
(v) U is CH₂;
V is O;
W is CH;
X is C—OH; and
R¹ and R² are both hydrogen.

37. The compound of claim 35, or a pharmaceutically acceptable salt thereof, wherein
(i) U is CH₂;
V is O;
W and X are both CH;
R¹ is selected from halogen and C₁₋₆-alkyl; and
R² is selected from hydrogen and halogen; or
(ii) U is CH₂;
V is NH;
W and X are both CH; and
R¹ and R² are both hydrogen; or
(iii) U and V together form a group C═C;
W and X together form a group C═C; and
R¹ and R² are both hydrogen.

38. The compound of claim 35, or a pharmaceutically acceptable salt thereof, wherein
(i) U is CH₂;
V is O;
W and X are both CH;
R¹ is selected from fluoro and methyl; and
R² is selected from hydrogen and fluoro; or
(ii) U is CH₂;
V is NH;
W and X are both CH; and
R¹ and R² are both hydrogen; or
(iii) U and V together form a group C═C;
W and X together form a group C═C; and
R¹ and R² are both hydrogen.

39. The compound of claim 35, or a pharmaceutically acceptable salt thereof, wherein:
Z is N;
Q is CH; and
m and n are both 0.

40. The compound of claim 35, or a pharmaceutically acceptable salt thereof, wherein L is a covalent bond, —CHR⁵—, or —CH₂O—.

41. The compound of claim 35, or a pharmaceutically acceptable salt thereof, wherein L is a covalent bond or —CH₂O—.

42. The compound of claim 35, or a pharmaceutically acceptable salt thereof, wherein:
A is C₆-C₁₄-aryl;
R³ is hydrogen or halo-C₁-C₆-alkyl; and
R⁴ is hydrogen or halogen.

43. The compound of claim 42, or a pharmaceutically acceptable salt thereof, wherein:
Z is N;
Q is CH;
m and n are both 0; and
L is a covalent bond, —CHR⁵—, or —CH₂O—.

44. The compound of claim 35, or a pharmaceutically acceptable salt thereof, wherein:
A is C₆-C₁₄-aryl;
R³ is halo-C₁-C₆-alkyl; and
R⁴ is hydrogen or halogen.

45. The compound of claim 35, or a pharmaceutically acceptable salt thereof, wherein:
A is phenyl;
R³ is CF₃ or 2,2,2-trifluoroethyl; and
R⁴ is hydrogen or fluoro.

46. The compound of claim 45, or a pharmaceutically acceptable salt thereof, wherein:
Z is N;
Q is CH;
m and n are both 0; and
L is a covalent bond or —CH₂O—.

47. The compound of claim 35, wherein the compound is:
rel-(4aR,8S,8aS)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one;
rel-(4aS,8R,8aR)-6-[3-[[2-Fluoro-4-(trifluoromethyl)phenyl]methoxy]azetidine-1-carbonyl]-8-methyl-4,4a,5,7,8,8a-hexahydropyrido[4,3-b][1,4]oxazin-3-one;
rel-(4aS,8aS)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one;
rel-(4aR,8aR)-8,8-Difluoro-6-[3-[[2-fluoro-4-(trifluoromethyl)phenyl]methoxy]azetidine-1-carbonyl]-4a,5,7,8a-tetrahydro-4H-pyrido[4,3-b][1,4]oxazin-3-one;
rel-(4aS,8aS)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one;
rel-(4aR,8aR)-8,8-Difluoro-6-[3-[4-(2,2,2-trifluoroethyl)phenyl]azetidine-1-carbonyl]-4a,5,7,8a-tetrahydro-4H-pyrido[4,3-b][1,4]oxazin-3-one;
6-[4-[[4-(Trifluoromethyl)phenyl]methyl]piperidine-1-carbonyl]-4,5,7,8-tetrahydropyrido[4,3-b][1,4]oxazin-3-one;
7-(4-Benzhydrylpiperidine-1-carbonyl)-1,5,6,8-tetrahydro-1,7-naphthyridin-2-one;
7-[3-[[2-Fluoro-4-(trifluoromethyl)phenyl]methoxy]azetidine-1-carbonyl]-1,5,6,8-tetrahydro-1,7-naphthyridin-2-one;
rac-(4aS,8aS)-7-(4-Benzhydrylpiperidine-1-carbonyl)-1,3,4,4a,5,6,8,8a-octahydro-1,7-naphthyridin-2-one;
rac-(4a5,8a5)-7-[3-[[2-Fluoro-4-(trifluoromethyl)phenyl]methoxy]azetidine-1-carbonyl]-1,3,4,4a,5,6,8,8a-octahydro-1,7-naphthyridin-2-one;
rac-(4aR,8aS)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one;
(4aR,8a5)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one;
(4a5,8aR)-6-[3-[[2-Fluoro-4-(trifluoromethyl)phenyl]methoxy]azetidine-1-carbonyl]-1,2,4,4a,5,7,8,8a-octahydropyrido[3,4-b]pyrazin-3-one; or
6-[3-[[2-Fluoro-4-(trifluoromethyl)phenyl]methoxy]azetidine-1-carbonyl]-1,2,4,5,7,8-hexahydropyrido[3,4-b]pyrazin-3-one;
or a pharmaceutically acceptable salt thereof.

48. A process of manufacturing a compound of claim 35, or pharmaceutically acceptable salt thereof, comprising:
(a) reacting an amine of formula 2, wherein m, n, Q, L, A, R³ and R⁴ are as defined in claim 35, 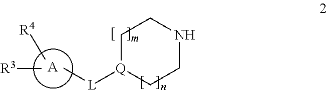
with a carboxylic acid 3a, wherein U, V, W, X, R¹ and R² are as defined in claim 35, 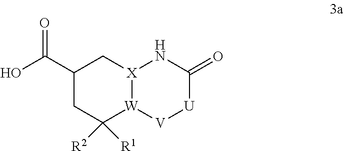
in the presence of a coupling reagent, and optionally in the presence of a base; or
(b) reacting an amine of formula 2, wherein m, n, Q, L, A, R³ and R⁴ are as defined in claim 35, 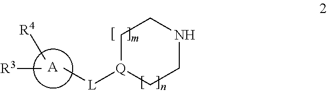
with a carboxylic acid chloride 3b, wherein U, V, W, X, R¹ and R² are as defined in claim 35, 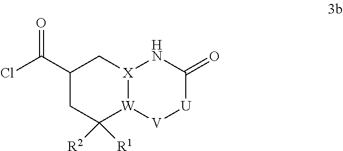
in the presence of a base; or
(c) reacting a first amine of formula 1, wherein U, V, W, X, R¹ and R² are as defined in claim 35, 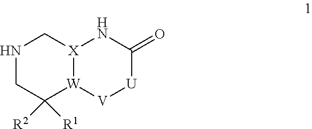
with a second amine 2, wherein A, L, m, n, Q, R³ and R⁴ are as defined in claim 35, 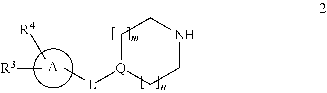
in the presence of a base and a urea forming reagent,
to form said compound of claim 35, or pharmaceutically acceptable salt thereof.

49. A pharmaceutical composition, comprising a compound of claim 35, or a pharmaceutically acceptable salt thereof, and a therapeutically inert carrier.

50. A pharmaceutical composition, comprising a compound of claim 47, or a pharmaceutically acceptable salt thereof, and a therapeutically inert carrier.

51. A method for the treatment or prophylaxis of a disease or disorder in a mammal, the method comprising administering an effective amount of a compound of claim 35, or a pharmaceutically acceptable salt thereof, to the mammal,
wherein the disease or disorder is neuroinflammation, neurodegenerative disease, pain, cancer, mental disorder, or inflammatory bowel disease in a mammal.

52. The method of claim 51, wherein the disease or disorder is multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain in a mammal, abdominal pain, abdominal pain associated with irritable bowel syndrome, or visceral pain.
